(19) United States
(12) Patent Application Publication
(10) Pub. No.: US 2026/0325659 A1
(43) Pub. Date: Oct. 22, 2026

(54) TREHALOSE SURFACED-DENDRIMER-DRUG CONJUGATES FOR THE TREATMENT OF OCULAR DISORDERS

(71) Applicants: Washington State University, Pullman, WA (US); Wayne State University, Detroit, MI (US)

(72) Inventors: Anjali Sharma, Pullman, WA (US); Rishi Sharma, Pullman, WA (US); Anu Rani, Pullman, WA (US); Anubhav Dhull, Pullman, WA (US); Nikhlesh Singh, Detroit, MI (US)

(21) Appl. No.: 19/458,270

(22) Filed: Jan. 23, 2026

Related U.S. Application Data
(60) Provisional application No. 63/750,203, filed on Jan. 27, 2025.
(60) Provisional application No. 63/855,206, filed on Jul. 31, 2025.

(51) Int. Cl. A61K 47/60; A61K 31/277; A61K 31/404; A61K 31/4439; A61K 31/4709; A61K 31/502; A61K 31/506; A61K 31/517; A61K 31/53; A61K 45/00; A61P 27/02

(52) U.S. Cl. CPC A61K 47/60; A61K 31/277; A61K 31/404; A61K 31/4439; A61K 31/4709; A61K 31/502; A61K 31/506; A61K 31/517; A61K 31/53; A61K 45/00; A61P 27/02

(57) ABSTRACT

Provided herein is a method for delivering one or more agents to a retina or cornea of a subject. The method includes administering to the subject a composition comprising a trehalose-surfaced dendrimer (Tre-D) conjugated to one or more agents, such as a VEGF inhibitor. The methods may include treatment of an ocular disorder in a subject in need thereof.

### CROSS-REFERENCE TO RELATED APPLICATIONS

This application claims benefit of United States provisional patent applications 63/750,203, filed Jan. 27, 2025 and 63/855,206 filed Jul. 31, 2025, the contents of which are incorporated herein by reference.

### STATEMENT OF FEDERALLY SPONSORED RESEARCH AND DEVELOPMENT

This invention was made with government support under grant number R01 EY029709 awarded by the National Institutes of Health. The government has certain rights in the invention.

### FIELD OF THE INVENTION

The invention is generally related to nanoparticle dendrimer compositions for targeted delivery of therapeutic agents, and treatment of ocular disorders to prevent vision loss.

### BACKGROUND OF THE INVENTION

Proliferative retinopathies, including diabetic retinopathy, age-related macular degeneration, and retinopathy of prematurity, are leading causes of vision loss worldwide. Current treatment paradigms rely heavily on intravitreal injections of anti-vascular endothelial growth factor A (anti-VEGFA) agents, which, despite their efficacy, are associated with ocular complications (endophthalmitis, retinal detachment, and elevated intraocular pressure), patient discomfort, and limited drug diffusion to neovascular tufts. Additionally, repeated injections can lead to systemic adverse effects, fibrosis, and tachyphylaxis. More effective alternative treatments are needed.

Proliferative retinopathy, also known as pathological retinal neovascularization (NV), represents a significant cause of moderate to severe vision loss across various age groups. This condition manifests in several forms, including retinopathy of prematurity (ROP) in infants, diabetic retinopathy (DR) in adults, and neovascular age-related macular degeneration (nAMD) in the elderly. The underlying mechanism of these diseases involves aberrant angiogenic responses to ischemia or hypoxia. However, this process often leads to pathological NV in the vitreous, resulting in interference with light transmission and subsequent vision impairment. This aberrant NV is driven by the high levels of vascular endothelial growth factor A (VEGFA), resulted due to hypoxia. One of the primary treatment modalities for advanced stages of retinopathy is laser photocoagulation. This technique effectively targets and destroys the abnormal blood vessels that contribute to further retinal damage. While laser treatment has proven effective in many cases, it is not without limitations. There are instances where the underlying pathological processes continue to progress despite treatment, emphasizing the urgent need for alternative therapeutic strategies.

The advent of intravitreal injections of anti-VEGFA agents has revolutionized the management of neovascular retinal diseases. These therapies specifically inhibit the action of VEGFA, a key player in the angiogenic process. VEGFA has been the focus of many current anti-angiogenic regimens because it is the most well-studied angiogenic and permeability factor involved in proliferative retinopathies. However, the intravitreal injection of anti-VEGF agents can be painful and carries risks of post-injection complications, such as elevated intraocular pressure, intraocular inflammation, and even endophthalmitis. Even after intravitreal delivery, anti-VEGF therapies face challenges in adequately reaching the sites of active angiogenesis, thereby limiting their therapeutic effectiveness.

Axitinib, a multi-receptor tyrosine kinase inhibitor (TKI), has potent inhibitory effects on vascular endothelial cell growth factor receptor 1-3 (VEGFR 1-3), platelet derived growth factor receptor beta (PDGFR-β) and KIT receptors. Axitinib was approved in 2012 by the United States Food and Drug Administration (FDA) for treatment of renal cell carcinoma. An injectable suspension of Axitinib is currently being investigated for the treatment of AMD using a suprachoroidal microinjector platform (NCT04626128). Multiple in vivo studies in mouse, rat, and rabbit models support the potential activity of Axitinib for the treatment of NV in retinal and choroidal tissues. Owing to its potent pan-VEGFR inhibition, Axitinib may provide a meaningful benefit compared to current anti-VEGFA agents, which lead to the upregulation of VEGF-C and VEGF-D. This upregulation of other VEGF ligands could contribute to tachyphylaxis, and may lead to refractory cases clinically. Despite the success of Axitinib and other VEGF TKIs in cancer treatments, its ophthalmic applications for NV are still impeded by its poor bioavailability in the posterior eye. Another reason Axitinib needs to be delivered precisely in the body because it can lead to side-effects including hypertension, and cerebrovascular ischemic events.

Currently, systemic and controlled release technologies are actively being explored to limit intravitreal injections and prolong the action of anti-VEGFA therapies. Nanotechnology offers a promising avenue for developing more effective treatment strategies by precise delivery of drugs to targeted locations. Various nano-sized delivery systems, including polymers, dendrimers, nanowires, microneedles, and lipid-based nanoparticles, have shown potential in delivering therapeutic agents directly to specific cells in the retina and cornea. However, the majority of these interventions still rely on intraocular injections or local delivery methods, indicating a need for further development and refinement in nanotechnology applications for the ocular therapies. Currently, no organic nanoparticles have been shown to selectively target aberrant neovascular tufts at retinal pathology sites following systemic administration.

### SUMMARY

An aspect of the disclosure provides a method for delivering one or more agents, e.g. a therapeutic agent, to a retina or cornea of a subject, comprising administering to the subject a composition comprising a trehalose-surfaced dendrimer (Tre-D) conjugated to the one or more agents. In some embodiments, the Tre-D targets aberrant neovascular tufts in a retina or aberrant blood vessels in a cornea of the subject. In some embodiments, the one or more agents are conjugated to an outer surface of the Tre-D. In some embodiments, the one or more agents are conjugated to the outer surface via a covalent bond or via noncovalent interactions. In some embodiments, the one or more agents comprises a vascular endothelial growth factor (VEGF) inhibitor. In some embodiments, the one or more agents comprises one or more of an anti-angiogenic agent, a tyrosine kinase inhibitor, an anti-cancer agent, a steroid, an anti-inflammatory agent (NSAID), and an anti-infective agent. In some embodiments, the tyrosine kinase inhibitor is one or more of axitinib, lenvatinib, dasatinib, tivozanib, cabozantinib, sorafenib, regorafenib, anlotinib, brivanib, su1498, vorolanib, vatalanib, pazopanib, and vandetanib. In some embodiments, the NSAID is one or more of aspirin, tolmetin, naproxen, bromfenac, ketorolac, nepafenac, ibuprofen, piroxicam, mefenamic acid, meloxicam, celecoxib, fenoprofen, sulindac, oxaprozin, etodolac, indomethacin, diclofenac, ketorolac, neparenac, and bromfenac. In some embodiments, the one or more therapeutic agents is present in the composition in an amount of 0.1-80 wt %. In some embodiments, the Tre-D is administered via systemic, intravitreal, suprachoroidal, periocular, subconjunctival, intracameral, intraperitoneal, topical, oral, transscleral, subretinal, or implantable device administration.

Another aspect of the disclosure provides a method for treating an ocular disorder in a subject in need thereof, comprising administering to the subject a therapeutically effective amount of Tre-D which has been demonstrated herein to have therapeutic efficacy without any additional active agent. In some embodiments, the Tre-D is conjugated to one or more additional therapeutic agents suitable for treating the ocular disorder. In some embodiments, the ocular disorder is selected from the group consisting of diabetic retinopathy, age related macular degeneration, proliferative retinopathies, retinitis pigmentosa, Stagardt's disorder, ocular histoplasmosis, choroidal neovascularization, corneal neovascularization, ocular inflammation uveitis, retinopathy of prematurity (ROP), retinal vein occlusion (RVO), hypertensive retinopathy, retinal angiomatomous proliferation, glaucoma, retinal detachment, and macula edema.

### BRIEF DESCRIPTION OF THE DRAWINGS

FIG. 1 depicts synthesis of Trehalose dendrimer (11). Reagent and conditions (i) DMF, NaH, RT, 3 h, 75% (ii) LiOH. H2O, THF-H2O, RT, 24 h, 78% (iii) CuSO4.H2O, Sodium ascorbate, DMF, 50 °C, 12 h, 90% (iv) EDC. HCl, HOBt, DCM, RT, 1.5 h 85% (v) NaOMe, MeOH, RT, 16 h, 92% (vi) CuSO4.5H2O, Sodium ascorbate, 40 °C, MW, 15 h, 85% (vii) NaOMe, MeOH, RT, 16 h, 90% (vi) CuSO4.5H2O, Sodium ascorbate, 80 °C, MW, 15 h, 86%.

FIG. 2A-C shows synthesis and characterization of Tre-D. (A) 1H NMR spectra of intermediate dendrons, hyper-core, protected and deprotected Tre-D dendrimers representing the appearance and disappearance of characteristic protons; (B) HPLC chromatogram of Tre-D showing >99% purity; (C) 1H NMR spectra and HPLC traces demonstrate reproducibility in the structure and purity among three different (5g) batches of Tre-D.

FIG. 3A-C shows synthesis and characterization of fluorescently labeled and unlabeled TreD-Axitinib conjugates. (A) Synthesis of Tre-D-Cy5, Axitinib azide, Tre-D-Axitinib and Tre-D-Axitinib-Cy5 conjugates, Reagents and conditions: (i) Pd(dppf)2Cl2.DCM, K3PO 4, DMF, 80 °C, 12 h, 82%; (ii) NaOH, MeOH: THF: H2O, RT, 2 h, 90%; (iii) HATU, DIPEA, DMF, RT, 1 h, 70%; (iv) TFA, 60 °C, 1 h, 75%; (v) 5-hexynoic acid, EDC. HCl, DMAP, DMF, 24 h, RT, 90%; (vi) Cy5-azide, CuSO4.5H2O, Sodium ascorbate, DMF: H2O, 40 °C, 10 h, 89%, (vii) CuSO4.5H2O, Sodium ascorbate, DMF: H2O, RT, 24 h, 86%; (viii) CuSO 4.5H2O, Sodium ascorbate, DMF: H2O, RT, 24 h, 88%; (B) 1H NMR stacked spectra of Tre-D-Hexyne (12), Tre-D-Cy5 (13), Axitinib-azide (19), Tre-D-Axitinib (20), and Tre-D-Axitinib-Cy5 (21) conjugate showing the appearance of characteristic protons; (C) HPLC chromatogram of Axitinib-azide, Tre-D-Hexyne and Tre-D-Axitinib, and Tre-D-Axitinib-Cy5 conjugates showing significant shift in retention time and >99% purity.

FIG. 4A-D shows physiochemical characterization of Tre-D-Axitinib conjugate. (A) Size and zeta potential distribution of Tre-D and Tre-D-Axitinib analyzed by dynamic light scattering (DLS) in triplicates; (B) MALDI-TOF of Tre-D-Axitinib; (C) Tre-D-Axitinib has several folds higher water solubility than free Axitinib; (D) Table representing physicochemical properties of Tre-D-Axitinib.

FIG. 5A-D is imaging supporting cellular uptake and VEGFR2 inhibition potential of Tre-D and Tre-D-Axitinib conjugates. Confocal micrographs illustrate the uptake of (A) Tre-D-Cy5 and (B) Tre-D-Axitinib-Cy5 dendrimer in HUVECs, indicated by the fluorescence of Cy5 within the cells. The scale bar represents 75 μm. Images are representative of three independent experiments. (C-D) IC₅₀ values for VEGFR2 inhibition are shown for (C) Tre-D and (D) Tre-D-Axitinib.

FIG. 6A-F shows effect of Tre-D-Axitinib on angiogenic events and cell migration in HUVECs. (A) In vitro cellular compatibility of HUVECs treated with Tre-D-Axitinib at different concentrations, (B) BrdU assay for determining the anti-cell proliferation efficacy of Tre-D-Axitinib on HUVECs. (C, D) Microscopy evaluation of anti-angiogenesis effect of Tre-D-Axitinib at differentiation concentrations and corresponding bar graph showing quantitative ImageJ analysis. (E, F) Quantitative ImageJ and microscopic evaluation by scratch assay, depicting the in vitro anti-migration effect of Tre-D-Axitinib at different concentrations. Data is representation of one of the triplicates and the scale bar represents 200 μm in C and F. For cell viability, proliferation and angiogenesis assay, the statistical significance was calculated using ordinary one-way ANOVA, and for cell migration assay, by two-way ANOVA, (****, p<0.0001; ***, p<0.001; **, p<0.01; ns=nonsignificant).

FIG. 7A-I shows retinal localization of Tre-D-Cy5 to neovascular tufts following systemic administration. (A) Schematic illustrating the mouse OIR model. C57BL/6 mouse pups with their dams were subjected to 75% oxygen from postnatal day 7 to postnatal day 12 and thereafter restored to room air on postnatal day 12. The P12-P17 period represents the neovascular phase. (B) C57BL/6 mice pups were exposed to OIR, at P12 the pups were returned to room air, and an IP injection of Tre-D-Cy5 (20 mg/kg Bwt) was given. At P15 eyes were enucleated, retinas isolated, stained with isolectin B4, flat mounts were made and examined for Tre-D-Cy5 in retinal vasculature. (C) C57BL/6 mice pups were exposed to normoxia or OIR, administered with Tre-D-Cy5 at P12, and at P15 eyes were enucleated, fixed, and embedded in OCT. The cryosections were prepared and stained with CD-31. (D) C57BL/6 mice pups were exposed to OIR. At P12 pups were given IP injection of Tre-D-Axitinib-Cy5 (20 mg/kg Bwt), and at P17 various organs were collected and organ biodistribution of Tre-D-Cy5 was calculated using fluorescence spectroscopy. Since Axitinib alone is not fluorescently labeled, it cannot be detected or quantified using the imaging approach employed in this experiment. Therefore, the inclusion of an Axitinib-alone group would not provide meaningful comparative data in the context of these specific analyses (FIG. 7C & D) and hence was not included as a control in these experiments. (E, F) Mice pups were exposed to OIR, and at P12 pups were given an IP injection of PBS (saline) and Tre-D-Axitinib-Cy5(20 mg/kg Bwt), and at P17 livers and kidneys were collected, fixed, cryosections were made and stained with hematoxylin & eosin (H&E) staining. (G-I) The serum ALT activity, AST activity, and creatinine levels were assessed in mouse pups (at P17) that were exposed to OIR and received an IP injection of either PBS or Tre-D-Axitinib-Cy5 at P12. Scale bar represents 50 μmin FIG. B & C, 20μm in far-right column of FIG. C, and 100 μm in FIG. E & F. NS, non-significant.

FIG. 8A-E shows Tre-D-Axitinib attenuates OIR-induced retinal NV. (A) Schematic showing the proposed experiments. (B) The C57BL/6 mice pups were exposed to OIR and, at P12, administered intraperitoneally with phosphate buffered saline (PBS), or 5, 10, or 20 mg/kg Bwt of Tre-D or Tre-D-Axitinib. At P17 eyes were enucleated, followed by the isolation of retinas. The retinas were then stained with isolectin B4, the flat mounts were prepared and examined for retinal NV. (C) The C57BL/6 mice pups were exposed to OIR and, at P12 administered intraperitoneally with PBS or various concentrations of Tre-D or Tre-D-Axitinib. At P17 eyes were enucleated, retinas isolated, stained with isolectin B4, flat mounts were made and examined for avascular area according to methods described by us previously. (D & E) The bar graphs represents NV (%) and avascular/total area (%) of 6 retinas from six individual animals. The values are presented as Mean±SD. * p<0.01 vs OIR+Tre-D. Scale bar is 500 μm in panel B, and C.

FIG. 9A-D shows Tre-D-Axitinib regulates VEGFA-induced angiogenic events in HRMVECs. (A) HRMVECs were treated with vehicle (PBS), Tre-D (20 ng/ml), or Tre-D-Axitinib (20 ng/ml or or 0.761 nM), followed by the administration of VEGFA at a concentration of 40 ng/mL for 24 h. The proliferation of HRMVECs was assessed using the BrdU proliferation assay. (B-D) Quiesced HRMVECs were first treated with Axitinib (50 M) or Tre-D-Axitinib (20 ng/mL or 0.761 nM) for 30 minutes and thereafter treated with or without VEGFA (40 ng/mL) and subjected to VEGF-induced migration (B), sprouting (C) and tube formation (D) according to methods described by us. The bar graphs or dot plots show the quantitative analysis of 3 to 6 independent experiments, expressed as mean±SD. Scale bar represents 20μm in figure C and 100 μmin figure D.

FIG. 10 is a graphical abstract providing an overview of the method of the Example.

### DETAILED DESCRIPTION

In the description herein, a word appearing in the singular encompasses its plural counterpart, and a word appearing in the plural encompasses its singular counterpart, unless implicitly or explicitly understood or stated otherwise. Furthermore, it is understood that for any given component or embodiment, any of the possible candidates or alternatives listed for that component may generally be used individually or in combination with one another, unless implicitly or explicitly understood or stated otherwise. Moreover, the figures are not necessarily drawn to scale, wherein some of the elements may be drawn merely for clarity. Also, reference numerals may be repeated among the various figures to show corresponding or analogous elements. Additionally, any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Various embodiments are described herein. In the following description, specific details of systems, components, and operations are included to provide a thorough understanding of certain embodiments of the disclosed technology. A person skilled in the relevant art will also understand that the technology may have additional embodiments. The technology may also be practiced without several of the details of the embodiments described below.

Embodiments of the disclosure provide compositions and methods for delivering one or more agents, e.g. a therapeutic agent or compound, to a retina or cornea of a subject, comprising administering to the subject a composition comprising a trehalose-surfaced dendrimer (Tre-D) conjugated to the one or more therapeutic agents. By eliminating the need for invasive intravitreal injections and addressing systemic toxicities, Tre-D introduces a systemic nanotherapeutic strategy with broad implications for treating ischemic retinopathies, thus preventing vision loss. In some embodiments, Tre-D alone may be used for treating an ocular disorder as described herein without conjugation to another therapeutic agent.

A dendrimer is a synthetic highly branched monodisperse and polyfunctional macromolecule, constituted by repetitive units (so-called “generations”) that are chemically bound to each other by an arborescent process around a multifunctional central core. Dendrimers can be considered to have three major portions: a core, an inner shell, and an outer shell. Exemplary chemical moieties for the core, ir shell, and outer shell are independently selected from dipentaerythritol, pentaerythritol, 2-(aminomethyl)-2-(hydroxymethyl) propane-1,3-diol, 2-ethyl-2-(hydroxymethyl) propane-1,3-diol,3,3′,3″,3′″-silanetetrayltetrakis (propane-1-thiol), 3,3-divinylpenta-1,4-diene, 3,3′,3″-nitrilotripropionic acid, 3,3′,3″-nitrilotris(N-(2-aminoethyl)propanamide), 3,3′,3″,3′″-(ethane-1,2-diylbis(azanetriyl)) tetrapropanamide, 3-(carboxymethyl)-3-hydroxypentanedioic acid, 2,2′-((2,2-bis((2-hydroxyethoxy)methyl) propane-1,3-diyl)bis(oxy))bis(ethan-1-ol), tetrakis(3-(trichlorosilyl) propyl)silane, 1-Thioglycerol, 2,2,4,4,6,6-hexachloro-1,3,5,215,415,615-triazatriphosphinine, 3-(hydroxymethyl)-5,5-dimethylhexane-2,4-diol, 4,4′,4″-(ethane-1,1,1-triyl)triphenol, 2,4,6-trichloro-1,3,5-triazine, 5-(hydroxymethyl) benzene-1,2,3-triol, 5-(hydroxymethyl)benzene-1,3-diol, 1,3,5-tris(dimethyl(vinyl)silyl)benzene, Carbosiloxane core, nitrilotrimethanol, ethylene diamine, propane-1,3-diamine, butane-1,4-diamine, 2,2′,2″-nitrilotris(ethan-1-ol), alpha cyclodextrin, beta cyclodextrin, gamma cyclodextrin, benzene-1,2,3,4,5,6-hexathiol, monosaccharide, disaccharides, trisaccharides, oligosaccharides, chitosan, and derivatives thereof.

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures. Generally, dendrimers have a diameter from about 1 nm up to about 50 nm, such as from about 1-5, 5-10, 10-15, 15-20, 20-25, 25-30, 35-40, 40-45 or 45-50 nm in diameter, including all single digits within these ranges.

Applications of dendrimers typically involve conjugating other chemical species to the dendrimer surface that can function as detecting agents (such as a dye molecule), affinity ligands, targeting components, radioligands, imaging agents, or pharmaceutically active compounds. In one embodiment, a trehalose-surfaced dendrimer includes a core; at least one interior layer comprising repeating units; and trehalose terminal surface groups attached to an outermost interior layer. The dendrimer may be a dendrimer as described in US 20250161463 incorporated herein by reference. In some embodiments, the entire periphery of the dendrimer consists of trehalose terminal groups. In some embodiments, the core comprises polyamidoamine, cyclodextrin, polylysine, 2,2-bismethylolpropionic, tetrazine, poly(propylene imine), polyethylene glycol, glycol, or adamantane, or molecules with multiple hydroxyl, carboxylic acid, alkynes, azides, amines, strained alkynes, tetrazine functional groups. In some embodiments, the at least one interior layer includes at least one focal point selected from triazole, gallic acid, an amino acid, a peptide, or a linear polymer. In some embodiments, the repeating units are selected from polyethylene glycol, an amino acid, a peptide, or a linear polymer. In some embodiments, the dendrimer includes 1 to 15 interior layers, more particularly 1 to 10 interior layers. In some embodiments, the dendrimer is a mixed layer dendrimer. A “mixed layer” dendrimer refers to a dendrimer having distinct layers with different building blocks for different generations as opposed to identical units.

In a further embodiment, the dendrimer may include one to ten generations, wherein at least one of generation 1 to generation 10 includes trehalose groups, wherein the —OH of the trehalose groups are not attached to any other moiety or atom. In other words, free (i.e., exposed) trehalose groups can be attached at any location on the dendrimer including the core, an interior layer or at the dendrimer molecule surface.

In an additional embodiment, there is disclosed herein a dendrimer that includes a plurality of generations, wherein generation 1 includes a polyamidoamine bearing hexyne arms, generation 2 includes a gallic acid building blocks-based layer; and at least one additional generation includes trehalose surface groups. In certain embodiments, generation 3, generation 4, or generation 5 includes the trehalose surface groups.

In some embodiments, the dendrimer can include a plurality of linkers on the core, each linker having from 1 to 50 hydrocarbon units. In some embodiments, the dendrimers can include a plurality of linkages in the dendrimer molecule backbone selected from disulfide, ester, ether, carbonate, carbamate, thiol, thioester, cathepsin sensitive, maleimidomethyl, thioether, hydrazine, glucuronide bond, hydrazides, N-alkyl, ethyl, hydroxymethyl, and amide.

Also disclosed herein are compositions that include the trehalose-surfaced dendrimers crosslinked with at least one other agent such as hydrogels, linear polymers, hyperbranched polymers, glycopolymers, biopolymers, or implants. Illustrative glycopolymers include hyaluronic acid, chitosan, and dextran.

Attachment of various agents to the dendrimer may be accomplished by (1) a covalent attachment or conjugation to the external surface of the dendrimer, e.g. to form a dendrimer prodrug, (2) ionic coordination to charged outer functional groups, or (3) micelle-like encapsulation of an agent via a dendrimer-drug supramolecular assembly.

The molecular weight of the dendrimers can be varied to prepare polymeric nanoparticles that form particles having properties, such as drug release rate, optimized for specific applications. The dendrimers can have a molecular weight of between about 150 Da and 1 MDa. In certain embodiments, the polymer has a molecular weight of between about 500 Da and about 100 kDa, more preferably between about 1 kDa and about 50 kDa, most preferably between about 1 kDa and about 20 kDa.

Dendrimers may be prepared by methods known in the art. Dendritic structures are mostly synthesized by two different approaches: divergent or convergent. Many other synthetic pathways exist, such as the orthogonal approach, accelerated approaches, the double-stage convergent method or the hypercore approach, the hypermonomer method or the branched monomer approach, the double exponential method; the orthogonal coupling method or the two-step approach, the two monomers approach, or the AB2-CD2 approach.

The core of the dendrimer, one or more branching units, one or more linkers/spacers, and/or one or more surface groups can be modified to allow conjugation to further functional groups (branching units, linkers/spacers, surface groups, etc.), monomers, targeting agents, and/or active agents via click chemistry. “Click chemistry” involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition) (or equivalent thereof) (or any active end group such as, for example, a primary amine end group, a hydroxyl end group, a carboxylic acid end group, a thiol end group, etc.) on the second moiety. Further chemistries include metal catalysed/non-metal catalysed click chemistries, Staudinger ligation, oxime, thiol-ene, thiol maleimide, Amine-carboxyl, thiol-thiol, and hydrazide-aldehyde chemistries.

The trehalose-surfaced dendrimers may be used to deliver one or more agents, e.g. a therapeutic agent or compound, to the retina or cornea of a subject. The agent may be conjugated to an outer surface of the dendrimer, for example, via a covalent bond (cleavable or non-cleavable) or via noncovalent interactions. In some embodiments, the one or more agents comprises a vascular endothelial growth factor (VEGF) inhibitor. The VEGF inhibitor may inhibit any one or more of the VEGF receptors (i.e. VEGFR-1, VEGFR-2, VEGFR-3, PDGFR, c-KIT). The methods described herein may prevent tachyphylaxis associated with current anti-VEGFA treatments by inhibiting VEGFR1-3, PDGFR-β, and KIT receptors.

In some embodiments, the one or more agents comprises one or more of an anti-angiogenic agent, a tyrosine kinase inhibitor, an anti-cancer agent, a steroid, an anti-inflammatory agent (NSAID), and an anti-infective agent. In some embodiments, the tyrosine kinase inhibitor is one or more of axitinib, lenvatinib, dasatinib, tivozanib, cabozantinib, sorafenib, regorafenib, anlotinib, brivanib, su1498, vorolanib, vatalanib, pazopanib, and vandetanib. The tyrosine kinase inhibitor may decrease the expression of a gene selected from the group consisting of; PDGF, PDGFR-α, PDGFR-β, EGF, EGFR, RAF-a, RAF-c, AKT, RAS, NFkB, HIF, bFGF, bFGFR, Her-2, c-Met, c-Myc and HGF.

In some embodiments, the NSAID is one or more of aspirin, tolmetin, naproxen, bromfenac, ketorolac, nepafenac, ibuprofen, piroxicam, mefenamic acid, meloxicam, celecoxib, fenoprofen, sulindac, oxaprozin, etodolac, indomethacin, diclofenac, ketorolac, neparenac, and bromfenac.

In some embodiments, the one or more therapeutic agents is present in the composition in an amount of 0.01-99 wt %, e.g. 0.1-80 wt %.

In some embodiments, in addition to a therapeutic agent, one or more agents, such as one or more imaging agents, radioligands, and/or additional drugs are conjugated to the dendrimers.

In some embodiments, the agents are attached to the dendrimer via a linking moiety that is designed to be cleaved in vivo. The linking moiety can be designed to be cleaved hydrolytically, enzymatically, or combinations thereof, so as to provide for the sustained release of the active agents in vivo. Both the composition of the linking moiety and its point of attachment to the active agent, are selected so that cleavage of the linking moiety releases either an active agent, or a suitable prodrug thereof. The composition of the linking moiety can also be selected in view of the desired release rate of the active agents.

In some embodiments, the linkers on the trehalose dendrimers and linkers attached to drugs/bioactive agents can be a polyethylene glycol (PEG) linker, peptide, aliphatic chain, aromatic linker, oligopeptide linker, saccharide, disaccharide, or oligosaccharide.

In some embodiments, the attachment occurs via one or more of disulfide, ester, ether, thioester, carbamate, carbonate, hydrazine, or amide linkages. In preferred embodiments, the attachment occurs via an appropriate spacer that provides a disulfide bridge between the agent and the dendrimer. In this case, the dendrimer complexes are capable of rapid release of the agent in vivo by thiol exchange reactions, under the reduced conditions found in body.

Linking moieties generally include one or more organic functional groups. Examples of suitable organic functional groups include secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH2O2C—, CHRO2C—), wherein R is an alkyl group, an aryl group, or a heterocyclic group. In general, the identity of the one or more organic functional groups within the linking moiety can be chosen in view of the desired release rate of the active agents.

In some embodiments, the dendrimer comprises a linker arm attached with hydroxyl groups of trehalose with an ester, ether, thioether, hydrazone, azo, glycosidic, oxidation, or phenolic ether linkage. Further linkages may feature an epoxide opening, sulfation, oximation, acetylation, a Diels Alder reaction, or the hydroxy on the trehalose may be modified to bring a new functionality for attachment of drug linker or direct drug. These linkers can be a pH cleavable linker or non-cleavable linkages. The linker may have a reactive group for further conjugation, such as acetylene, tetrazine, amine, COOH, alcohol, thiol, aldehyde, azide, isocyanate, epoxy groups, carbamate, Dibenzocyclooctyne, Trans-Cyclooctene, Norbornene, Isonitrile, tetrazine, activated carboxylic acid, halogens, oxirane, aryl halide, olefin, vinyl, acrylate, isocyanide, diene, dienophile, sulfonic acid, nitrile group, silyl group, trimethylsilyl, tosylate, anhydride, ketone, formyl, or carbonyl group which can participate in reductive amination.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo-and polyethylene glycol chains, and oligo-and poly(amino acid) chains.

A wide range of agents may be associated with the dendrimers disclosed herein. The agents can be proteins or peptides, sugars or carbohydrate, nucleic acids or oligonucleotides, lipids, small molecules, aptamers, cytokines, steroids, hormones, or combinations thereof. The nucleic acid can be an oligonucleotide encoding a protein, for example, a DNA expression cassette or an mRNA. Representative oligonucleotides include siRNAs, microRNAs, shRNAs, DNA, and RNA. In some embodiments, the active agent is a therapeutic antibody, e.g. a monoclonal antibody such as Ranibizumab (Lucentis®), Bevacizumab (Avastin®) or an antibody fragment such as razumab. The active agent may be a fusion proteins such as Aflibercept (Eylea®), single-chain variable fragment such as Brolucizumab (Beovu®), a recombinant fusion protein composed of VEGFR-1 (second domain) and VEGFR-2 (third and fourth domains) regions fused to the Fc portion of human IgG1 immunoglobulin such as Conbercept (Lumitin®), etc. One or more types of active agents can be encapsulated, complexed or conjugated to the dendrimer.

In some embodiments, the dendrimer is linked to the agent via a spacer ending in disulfide, ester, ether, thioester, carbamate, carbonate, hydrazine, or amide bonds. Generally, one or more prophylactic, therapeutic, and/or diagnostic agents are encapsulated, associated, and/or conjugated in the dendrimer complex at a concentration of about 0.01% to about 90%, preferably about 0.1% to about 80%, by weight. The dendrimer can be conjugated to more than one agent and more than one type of agent.

The trehalose-surfaced dendrimers described herein are particularly useful for targeting neovascular tufts in the retina or aberrant blood vessels in the cornea which can inhibit pathological angiogenesis. The trehalose-surfaced dendrimer alone or the dendrimer-drug conjugates can reverse or reduce the aberrant neovascularization and proliferation of neovascular tissues, tuft formation, and vessel anastomoses in various ocular disorders. Embodiments provide methods of treating an ocular disorder, or more particularly, a retinal disorder, in a subject in need thereof comprising administering to the subject a therapeutically effective amount of a trehalose-surfaced dendrimer as described herein. In some embodiments, the dendrimer may be conjugated to one or more therapeutic/bioactive agents suitable for treating the ocular disorder. In some embodiments, the ocular disorder is selected from the group consisting of diabetic retinopathy, age related macular degeneration, proliferative retinopathies, retinitis pigmentosa, Stagardt's disorder, ocular histoplasmosis, choroidal neovascularization, corneal neovascularization, ocular inflammation uveitis, retinopathy of prematurity (ROP), retinal vein occlusion (RVO), hypertensive retinopathy, retinal angiomatomous proliferation, glaucoma, retinal detachment, and macula edema.

As used herein “treating” or “treatment” means (a) inhibiting the disease, i.e. slowing or halting the development of clinical symptoms; and/or (b) alleviating the disease, i.e. causing regression of clinical symptoms and/or or (c) any treatment of the disease, including alleviation or elimination of the disease and/or its attendant symptoms.

The methods described herein minimize systemic adverse effects associated with free anti-angiogenic drugs, including hypertension and cerebrovascular ischemic events.

In some embodiments, the methods described herein include an initial step of determining if the subject has an ocular disorder as described herein and if the subject is determined to have the ocular disorder, then administering to the subject a therapeutically effective dose of a composition as disclosed herein.

The compositions described herein may be administered in vivo by any suitable route (e.g. parenterally or enterally) including but not limited to: inoculation or injection (e.g. intravenous, intraperitoneal, intramuscular, subcutaneous, intra-aural, intraarticular, intramammary, and the like), topical application, and by absorption through epithelial or mucocutaneous linings (e.g., nasal, oral, vaginal, rectal, gastrointestinal mucosa, and the like). Other suitable means include but are not limited to: inhalation (e.g. as a mist or spray), orally (e.g. as a pill, capsule, liquid, etc.), intravaginally, intranasally, rectally, by ingestion of a food or probiotic product containing the compound, as eye drops, etc. In preferred embodiments, the mode of administration systemic, intravitreal, suprachoroidal, periocular, subconjunctival, intracameral, intraperitoneal, topical, oral, transscleral, subretinal, or implantable device administration.

As used herein, “retina” includes the inner retina and the outer retina as well as supporting and ancillary structures within the eye including, without limitation, the choroid, choriocapillaris, and retinal pigment epithelium. The methods described herein also can be used to effectively deliver an agent to other (e.g., non-retinal) structures in the eye such as the pars plana, ciliary processes, ciliary body, the iris, the lens, the trabecular meshwork, and/or the cornea or corneal endothelium.

As used herein, “cornea” refers to the convex, transparent anterior part of the eye, comprising one sixth of the outermost tunic of the eye bulb. It allows light to pass through it to the lens. The cornea is a fibrous structure with five layers: the anterior corneal epithelium, continuous with that of the conjunctiva; the anterior limiting layer (Bowman's membrane); the substantial propria; the posterior limiting layer (Descemet's membrane); and the endothelium of the anterior chamber (keratoderma). It is dense, uniform in thickness, and nonvascular, and it projects like a dome beyond the sclera, which forms the other five sixths of the eye's outermost tunic.

A patient or subject to be treated by any of the compositions or methods of the present disclosure can mean either a human or a non-human animal including, but not limited to mammals, dogs, horses, cats, rabbits, gerbils, hamsters, rodents, birds, aquatic mammals, cattle, pigs, camelids, and other zoological animals.

In some embodiments, the formulation or active agent is administered to the subject in a therapeutically effective amount. By a “therapeutically effective amount” or an “effective amount” is meant a sufficient amount to treat the disease or disorder at a reasonable benefit/risk ratio applicable to any medical treatment. It will be understood that the total daily usage of the compositions of the present disclosure will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. It is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 1 to 1,500 mg per adult per day. In particular, the compositions contain at least or up to 1.0, 2.5, 5.0, 10.0, 15.0, 25.0, 50.0, 100, 250, 500, 750, 1000, 1250, or 1500 mg of the active ingredient for the symptomatic adjustment of the dosage to the subject to be treated. A medicament typically contains from about 0.01 mg to about 1500 mg of the active ingredient, in particular from 1 mg to about 250 mg of the active ingredient. An effective amount of the drug is ordinarily supplied at a dosage level at least or up to 1 mg/kg to 100 mg/kg of body weight per day, e.g. about 5 mg/kg, 10 mg/kg, 15 mg/kg, 20 mg/kg, 30 mg/kg, 40 mg/kg, 50 mg/kg, 60 mg/kg, 70 mg/kg, 80 mg/kg, 90 mg/kg, or 100 mg/kg of body weight per day. Such doses may be administered in a single dose or it may be divided into multiple doses.

Embodiments of the disclosure also provide pharmaceutical compositions comprising the dendrimer complexes described herein and a pharmaceutically acceptable carrier/excipient. The pharmaceutical compositions can be formulated according to known methods for preparing pharmaceutically useful compositions. The active ingredients may be mixed with excipients which are pharmaceutically acceptable and compatible with the active ingredients. “Pharmaceutically” or “pharmaceutically acceptable” refer to molecular entities and compositions that do not produce an adverse, allergic or other untoward reaction when administered to a mammal, e.g. a human, as appropriate. As used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19th ed.) describes formulations which can be used in connection with the subject invention. The final amount of the compounds in the formulations may vary. However, in general, the amount in the formulations will be from about 0.01-99%, weight/volume.

The dendrimer complexes described herein include the pharmaceutically acceptable salts thereof. “Salts” or “pharmaceutically acceptable salts” refers to the relatively non-toxic, inorganic and organic acid addition salts, and base addition salts, of compounds of the present disclosure. These salts can be prepared in situ during the final isolation and purification of the compounds. In particular, acid addition salts can be prepared by separately reacting the purified compound in its free base form with a suitable organic or inorganic acid and isolating the salt thus formed.

Compositions as described herein may be prepared either as liquid solutions or suspensions, or as solid forms such as tablets, pills, granules, capsules, powders, ampoules, and the like. The liquid may be an aqueous liquid. Solid forms suitable for solution in, or suspension in, liquids prior to administration may also be prepared. Example dosage forms include a tablet, dragee, liquid, drop, capsule, caplet, gelcap, etc.

Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question. The pharmaceutical composition can be adapted for various forms of administration. Administration can be continuous or at distinct intervals as can be determined by a person skilled in the art.

The compositions of the present disclosure may also contain other components such as, but not limited to, additives, adjuvants, buffers, tonicity agents, bioadhesive polymers, and preservatives. In any of the compositions of this disclosure, the mixtures are preferably formulated at about pH 5 to about pH 8. This pH range may be achieved by the addition of buffers to the composition. It should be appreciated that the compositions of the present disclosure may be buffered by any common buffer system such as phosphate, borate, acetate, citrate, carbonate and borate-polyol complexes, with the pH and osmolality adjusted in accordance with well-known techniques to proper physiological values.

An additive such as a sugar, a glycerol, and other sugar alcohols, can be included in the compositions of the present disclosure. Pharmaceutical additives can be added to increase the efficacy or potency of other ingredients in the composition. For example, a pharmaceutical additive can be added to a composition of the present disclosure to improve the stability of the bioactive agent, to adjust the osmolality of the composition, to adjust the viscosity of the composition, or for another reason, such as effecting drug delivery. Non-limiting examples of pharmaceutical additives of the present disclosure include sugars, such as, trehalose, mannose, D-galactose, and lactose.

In an embodiment, if a preservative is desired, the compositions may optionally be preserved with any well-known system such as benzyl alcohol with/without EDTA, benzalkonium chloride, chlorhexidine, Cosmocil® CQ, or Dowicil 200.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLE

Here, we report a unique mixed layered trehalose-surfaced dendrimer (Tre-D), that is composed of biocompatible building blocks and selectively targets aberrant neovascular tufts when systemically administered in a mouse model of oxygen induced retinopathy (OIR), with no presence in normal retinal blood vessels. We further conjugate Axitinib on the surface of Tre-D to develop Tre-D-Axitinib conjugate. Our findings indicate that a single intraperitoneal injection of Tre-D-Axitinib can help attenuate abnormal blood vessel growth or pathological angiogenesis in ischemic retinopathies by selectively targeting neovascular tufts (FIG. 10). This indicates better non-invasive treatment options for proliferative retinopathies. This targeted approach maximizes Axitinib's efficacy while minimizing systemic side effects associated with the free drug. In addition, it provides a new set of treatment options that not only lessen the side effects of intravitreal injections but also those of anti-VEGFA therapies.

### Materials and Methods

### Chemistry-Experimental Section

### Material and Reagents

All starting materials and reagents were sourced from Sigma-Aldrich and Merck. Commercially available reagents and solvents used for the synthesis were of analytical grade and were used without further purification. Reactions requiring anhydrous conditions were carried out under a positive nitrogen flow, with all glassware dried in an oven. Analytical thin-layer chromatography (TLC) was performed using silica gel 60 F254 plates (aluminum foil), with spot visualization achieved via UV light or staining reagents. Compounds were isolated and purified through flash column chromatography on silica gel 60 (230-400 mesh). Spectra/Por dialysis membranes were obtained from Repligen,

### Instruments

Nuclear Magnetic Resonance (NMR) spectra were recorded on a Bruker 500 MHz high-resolution NMR spectrometer. The samples were prepared in deuterated solvents, including chloroform (CDCl3), deuterated DMSO (DMSO-d6) or deuterated water (D2°). Chemical shifts for 1H-NMR are reported in parts per million (ppm) referenced to the solvent peak set. The coupling constants (J) are provided in hertz (Hz)and the following abbreviations are used for the description of the patterns: s=singlet, d=doublet, t=triplet, q=quartet, m=multiplet. Microwave reactions were performed in a Biotage® Initiator+instrument using a sealed 10 mL/20 mL process vials. Reaction times refer to irradiation time at the target temperature, not the total irradiation time. The temperature was measured with an IR sensor. High resolution mass spectra were performed using ESI by direct infusion on a Bruker MALDI-TOF (trans-2-[3-(4-tert-Butylphenyl)-2-methyl-2-propenylidene]malononitrile matrix), and Bruker Q-TOF.

Particle size and zeta potential were determined by Dynamic Light Scattering (DLS) using a Malvern Zetasizer® Nano 90 (Westborough, MA) at 25 °C. To determine particle size, Tre-D or Tre-D-Axitinib was dissolved in deionized water (18.2 Ω) to a final concentration of 0.5 mg/mL. The solution was filtered through 0.2 μm syringe filters (Pall Corporation, HT Tuffryn membrane, 0.2 μm) directly into a UV-transparent disposable cuvette (dimensions: 12.5×12.5×45 mm). For zeta potential measurements, a sample at a concentration of 0.2 mg/mL in 10 mM NaCl was prepared using the same procedure. Zeta potential measurements were carried out using a Malvern Zetasizer® Nanoseries disposable folded capillary cell.

High-performance liquid chromatography (HPLC) was used to determine the purity of small molecules as well as dendrimer and dendrimer-drug conjugates. The stability studies were also evaluated using HPLC. The analyses were carried out using a Waters Acquity Arc® system (Milford, MA, USA), equipped with binary pumps, 2998 PDA detector, and a 2475 fluorescence detector. The analyses were performed using Waters Empower software. The samples were run using Waters C18 symmetry 300, 5 μm, 4.6×250 mm column using a gradient flow method starting with 90:10 (Solvent A: 0.1% TFA and 5% ACN in water; Solvent B: 0.1% TFA in ACN), gradually increasing to 40:60 (A: B) at 15 minutes, maintained at 40:60 (A: B) at 30 minutes and then returned to 90:10 (A: B) at 35 minutes. A flow rate of 1 mL/min was maintained during the run. The dendrimers and drug conjugates were monitored at 210 and 254 nm. The Cy5 conjugates were monitored at 650 nm.

### Synthetic Procedures

Synthesis of methyl 3,4,5-tris((3,6,9,12-tetraoxapentadec-14-yn-1-yl)oxy)benzoate (2): Compound 1 (2 g, 1.0 eq, 2.81 mmol) was dissolved in anhydrous DMF (20 mL). To the stirring solution, sodium hydride (505 mg, 4.5 eq, 12.64 mmol, 60% dispersion in mineral oil) was slowly added in portions at 0 °C. The solution was stirred for 15 min at 0 °C. This was followed by the addition of propargyl bromide (1.06 mL, 4.0 eq, 11.22 mmol, 80% w/w solution in toluene) at 0 °C, and the stirring continued at room temperature for another 3 h. On completion, the reaction was quenched by the addition of saturated ammonium chloride solution and extracted with ethyl acetate (2×150 ml). The combined organic layer was washed with chilled brine (2×100 ml), dried over Na2SO4, and evaporated in vacuo. The crude product was purified by silica flash column chromatography to afford compound 2 in 75% yield (1.7g) as a brown viscous liquid. 1H NMR (500 MHz, CDCl3) δ 7.29 (s, 2H), 4.17-4.25 (m, 12H), 3.84-3.90 (m, 7H), 3.78 (t, J=5.2 Hz, 3H), 3.63-3.72 (m, 35H), and 2.43 (s, 3H). 13C NMR (125 MHz, DMSO-d6) δ 166.3, 152.5, 142.3, 124.8, 108.5, 80.8, 77.6, 77.5, 72.8, 72.4, 70.5, 70.4, 70.3, 70.2, 70.1, 70.0, 69.4, 69.0, 68.9, 60.6, 57.94, 52.6. (MALDI-TOF) m/z: calculated for C41H62O 17 [M+Na]+: 849.3885; found 849.3870.

Synthesis of 3,4,5-tris((3,6,9,12-tetraoxapentadec-14-yn-1-yl)oxy)benzoic acid (3): To a stirring solution of compound 2 (1 g, 1.0 eq, 1.21 mmol) in THF (10 mL) was added a solution of LiOH. monohydrate (290 mg, 10 eq, 12.10 mmol) in DI water (4 mL). The reaction mixture was stirred at room temperature for 24 h. Progress was monitored with thin-layer chromatography (TLC). The reaction mixture was diluted with water (150 mL), acidified (pH 3-4) by the addition of saturated citric acid solution and extracted with DCM (2×100 mL). The combined organic layer was washed brine (50 mL), then dried using anhydrous Na2SO4, filtered, and evaporated in vacuo. The crude product was purified by silica flash column chromatography to afford compound 3 in 78% yield (780 mg) as a brown viscous liquid. 1H NMR (500 MHz, CDCl3) δ 7.14 (s, 2H), 4.05-4.14 (m, 12H), 3.73 (t, J=5.1 Hz, 4H), 3.67 (t, J=5.1 Hz, 3H), 3.49-3.59 (m, 35H), and 2.31 (m, 3H). 13C NMR (125 MHz, CDCl3) δ 169.9, 152.3, 143.2, 124.2, 109.7, 79.7, 79.6, 74.7, 74.6, 72.5, 70.9, 70.8, 70.7, 70.6, 70.6S, 70.5, 70.4, 70.3, 69.7, 69.1, 68.9, 58.4, 58.3. (MALDI-TOF) m/z: calculated for C40H60O17 [M-H]+: 811.3752; found 811.3752.

Synthesis of (2R,3R,4S,5R,6R)-2-(acetoxymethyl)-6-(((2R,3R,4S,5R,6S)-3,4,5-triacetoxy-6-(azidomethyl)tetrahydro-2H-pyran-2-yl)oxy)tetrahydro-2H-pyran-3,4,5-triyl triacetate (4): The compound (2R,3R,4S,5R,6R)-2-(acetoxymethyl)-6-(((2R,3R,4S,5S,6R)-3,4, 5-triacetoxy-6-(bromomethyl)tetrahydro-2H-pyran-2-yl)oxy)tetrahydro-2H-pyran-3,4,5-triyl triacetate (18.66 g, 26.63 mmol) was dissolved in dry DMF (280 mL) and stirred under a nitrogen environment. Solid sodium azide (8.69 g, 133.63 mmol) was added to the reaction mixture and the solution was heated at 55 °C and stirred for 16 h. At completion of reaction, the solvent was removed under reduced pressure and the crude was dissolved in EtOAc (500 mL). The organic layer was washed 3×150 mL with water and (3×100 mL) brine. The organic layer was dried with anhydrous Na2SO4 and concentrated under reduced pressure. The crude brown-solid was purified by column chromatography on silica gel to obtain 12.20 g of pure 4 as a white solid with 69.1% yield (12.2g) as a white solid. 1H NMR (500 MHz, DMSO-d6) δ 5.20-5.38 (m, 4H), 4.97-5.09 (m, 4H), 4.16-4.23 (m, 1H), 3.94-4.06 (m, 3H), 3.47-3.54 (m, 1H), 3.39-3.46 (m, 1H), and 1.98-2.10 (m, 21H). 13C NMR (125 MHz, DMSO-d6) δ 170.5, 170.2, 170.0, 169.8, 92.5, 92.4, 79.6, 70.0, 69.6, 69.5, 69.1, 68.5, 68.4, 62.2, 50.3, 40.4, 40.2, 40.1, 20.9, 20.9, 20.8, 20.8.

Synthesis of compound 5: To a stirred solution of compound 3 (614 mg, 1.0 eq, 0.75 mmol) in 5 mL of DMF in a microwave vial was added the solution of compound 4 (1.7 g, 3.5 eq, 2.57 mmol) dissolved in 5 mL of DMF. It was followed by the addition of CuSO4.5H2O (5 mol % per acetylene) dissolved in 1 mL of deionized water. After stirring for 2 minutes, sodium ascorbate (10 mol % per acetylene) solution in 0.5 mL of deionized water was added to the reaction vial and was irradiated in a microwave at 50 °C for 12 h. Progress of the reaction was monitored with TLC. After complete conversion, the reaction mixture was diluted with DCM (100 mL) and washed with saturated EDTA solution (7×30 ml) and brine (1×50 ml), then dried (Na2SO 4), filtered, and evaporated in vacuo. The crude product was purified by silica flash column chromatography to afford compound 5 as white solid in 90% yield (1.8 g). 1H NMR (500 MHz, DMSO-d6) δ 8.00 (s, 3H), 7.21 (s, 2H), 5.28 (m, 10H), 5.01 (m, 14H), 4.84 (m, 3H), 4.65 (d, J=14.4 Hz, 3H), 4.50 (m, 10H), 4.17 (m, 9H), 4.10 (m, 6H), 3.98 (m, 8H), 3.75 (m, 10H), 3.66 (t, J=4.8 Hz, 2H), 3.60 (m, 4H), 3.53 (m, 29H), 2.05 (m, 20H), and 1.98 (m, 38H). 13C NMR (125 MHz, CDCl3) δ 170.7, 170.6, 170.0, 169.9, 169.8, 169.7, 169.6, 169.5, 169.4, 162.6, 145.5, 92.2, 91.5, 72.3, 70.9, 70.8, 70.7, 70.6, 70.5, 70.4, 70.0, 69.9, 69.8, 69.7, 68.9, 68.5, 68.3, 61.7, 36.5, 31.4, 20.9, 20.8, 20.7, 20.6, 20.5. (MALDI-TOF) m/z: calculated for C120H167N9O70 [M-H]+2795.97 found 2794.97.

Synthesis of compound 7: Compound 5 (200 mg, 1.0 eq, 0.08 mmol) was dissolved in 4 mL of anhydrous DCM and stirred at 0 °C. EDC. HCl (22.54 mg, 1.5 eq, 0.12 mmol) and HOBT (16.72 mg, 1.4 eq, 0.11 mmol) were added to the reaction mixture and the reaction mixture was stirred for 30 minutes. To this stirring solution, compound 6 (47.55 mg, 2.0 eq, 0.16 mmol) in 1 mL of dry DCM was added. The reaction mixture was allowed to stir at room temperature for 1.5 h. Progress of the reaction was monitored with the help of TLC using UV light. At the completion of reaction, the reaction mixture was diluted with DCM and the organic layer was washed with water (5×30 ml) and brine (2×30 ml), then dried using anhydrous Na2SO4, followed by filtration and evaporation under reduced pressure. The crude product was purified by silica flash column chromatography to afford compound 7 (178mg) as white solid in 85% yield. 1H NMR (500 MHz, DMSO-d6) δ 8.46 (t, 1H), 8.00 (s, 3H), 7.18 (s, 2H), 5.27 (m, 10H), 5.00 (m, 13H), 4.84 (d, J=3.6 Hz, 3H), 4.65 (dd, J=14.5, 2.9 Hz, 4H), 4.50 (d, J=16.9 Hz, 10H), 4.16 (m, 12H), 3.98 (m, 10H), 3.75 (m, 6H), 3.66 (t, J=5.0 Hz, 2H), 3.51 (m, 53H), and 1.97 (m, 63H). 13C NMR (125 MHz, CDCl3) 175.2, 175.0, 174.9, 174.7, 174.6, 174.5, 170.7, 156.9, 149.2, 145.1, 134.5, 130.2, 111.5, 96.5, 96.2, 77.0, 75.2, 75.1, 75.0, 74.9, 74.8, 74.7, 74.6, 74.4, 74.3, 74.2, 74.1, 74.0, 73.8, 73.5, 73.4, 73.2, 73.1, 68.5, 66.9, 55.2, 54.8, 25.7, 25.6, 25.5, 25.5, 25.4.

Synthesis of compound 8: To a stirred solution of compound 7 (1 g, 1.0 eq, 0.32 mmol) dissolved in dry MeOH (10 mL), a dropwise addition of sodium methoxide (300 μL, 1.1 M solution in MeOH) was done to adjust the pH approximately 8.5-9. The reaction mixture was stirred overnight at room temperature. The completion of the reaction was monitored by TLC. On completion, the pH was adjusted between 6 to 7 with an Amberlite IR-120 resin. The resin was removed by filtration, and the solvent was removed by rotary evaporation to yield compound 8 in 92% yield (655 mg). 1H NMR (500 MHz, D2O) δ 8.05 (m, 3H), 7.16 (m, 2H), 5.09 (m, 3H), 4.65 (m, 12H), 4.26 (m, 4H), 4.11 (m, 4H), 3.93 (m, 4H), 3.74 (m, 90H), and 3.35 (m, 6H). 13C NMR (125 MHz, D2O) δ 169.3, 151.9, 143.7, 139.6, 126.0, 106.3, 93.3, 93.1, 72.5, 72.3, 72.1, 70.9, 70.8, 70.2, 70.1, 69.9, 69.8, 69.7, 69.6, 69.5, 69.4, 69.1, 68.8, 68.3, 62.9, 60.4, 50.7, 50.0, 39.6.

Synthesis of compound 9: G1 PAMAM dendrimer (1.1 g, 1.0 eq, 0.77 mmol) was dissolved in 5mL of anhydrous DMF under nitrogen atmosphere. In a separate glass vial, 5-hexynoic acid (2 g, 24 eq, 18.48 mmol) was dissolved in anhydrous DMF (5 mL), activated by adding EDC (3.5 g, 24 eq, 18.48 mmol) and stirred for 15 minutes. The activated hexynoic acid solution was then added dropwise to the G1 PAMAM dendrimer solution under continuous stirring followed by addition of DMAP (1.1 g, 12 eq, 9.24 mmol). The reaction mixture was left on stirring for 24 h at room temperature. Upon completion, the dialysis was performed using a 1 kDa cut-off dialysis membrane in DMF for 12 h. The final dialysis was performed against pure water. The resulting aqueous solution was lyophilized to afford compound 9 in 84% yield (1.4g). 1H NMR (500 MHz, DMSO-d6) δ 7.61-8.21 (m, 20H), 3.33 (s, 11H), 3.17 (s, 14H), 2.54-3.02 (m, 39H), 2.37-2.48 (m, 13H), 2.01-2.34 (m, 63H), and 1.62-1.70 (m, 16H). 13C NMR (125 MHz, DMSO-d6) δ 172.1, 172.0, 171.7, 84.5, 71.9, 55.4, 52.6, 50.1, 50.0, 38.8, 38.7, 37.4, 34.6, 33.7, 24.6, 17.9.

Synthesis of compound 10: To a stirred solution of compound 9 (7.0 mg, 1.0 eq, 0.0032 mmol) in DMF (1 mL) in a microwave vial was added a solution of compound 7 (100 mg, 10.0 eq, 0.033 mmol) dissolved in DMF (2 mL). To this solution, CuSO4.5H2O (5 mol % per acetylene) and sodium ascorbate (10 mol % per acetylene) dissolved in minimum amount of deionized water were added. The reaction vial was irradiated using microwave heating at 40 °C for 15 h. Formation of desired product was observed via HPLC analysis. Upon completion, the reaction mixture was transferred to a 3.5 kDa dialysis membrane and the dialysis was performed in DMF for 12 h. The final dialysis was performed in deionized water to exchange DMF. The aqueous solution was lyophilized to afford acetylated-trehalose (10) dendrimer in 85% yield. 1H NMR (500 MHz, DMSO-d6) δ 8.41 (t, J=5.8 Hz, Tre-D-amides), 7.94 (m, Tre-D-amides+triazole H), 7.85 (m, 3H), 7.75 (s, 4H), 7.11 (s, 16H, Tre-D-Ar-H), 5.21 (m, sugar-H), 4.94 (m, sugar-H), 4.78 (d, J=4.0 Hz, sugar-H), 4.58 (dd, J=14.5, 2.9 Hz, sugar-H), 4.41 (m, sugar-H), 4.10 (m, sugar-H), 3.96 (m, sugar-H+PEG-H), 3.87 (m, sugar-H+PEG-H), 3.69 (m, sugar-H+core+PEG), 3.59 (t, J=5.0 Hz, sugar-H+core+PEG), 3.51 (m, sugar-H+core), 3.43 (m, PEG-H), 3.00 (s, core-H), 1.91 (m, acetates), 1.17 (s, sugar-H). 13C NMR (125 MHz, DMSO-d6) δ 170.6, 170.2, 170.0, 152.2, 124.7, 106.7, 96.6, 72.6, 70.5, 70.4, 70.3, 70.2, 70.13, 70.1, 70.0, 69.8, 69.4, 69.3, 69.1, 68.9, 68.8, 67.7, 66.6, 63.9, 62.5, 49.7, 34.9, 21.1, 21.0, 20.9

Synthesis of compound 11
Route 1: To a stirred solution of acetylated-trehalose dendrimer 10 (1.0 g, 1.0 eq, 0.32 mmol) in dry MeOH (10 mL), sodium methoxide (300 μL, 1.1 M solution in MeOH) was added dropwise to adjust the pH approximately 8.5-9. The reaction mixture was stirred overnight at room temperature. The completion of the reaction was monitored by TLC. On completion, the pH was adjusted between 6 and 7 with an Amberlite IR-120 resin. The resin was removed by filtration, and the solvent was removed by rotary evaporation to yield trehalose-dendrimer 11 in 90% yield.
Route 2: To a stirred solution of compound 9 (26.6 mg, 1.0 eq, 0.012 mmol) in DMF (2mL) in a microwave vial was added the solution of compound 8 (230 mg, 10.0 eq, 0.12 mmol) dissolved in deionized water (3 mL). To this solution, CuSO4.5H2O (5 mol% per acetylene) and sodium ascorbate (10 mol % per acetylene) dissolved in minimum amount of deionized water were added. The reaction vial was irradiated using microwave heating at 80 °C for 15 h. The formation of the product was observed with the help of HPLC. Upon completion, the dialysis was performed to purify the desired dendrimer using a 3.5 kDa dialysis membrane in DMF for 12 h. The final dialysis was performed in DI water. The aqueous solution was lyophilized to afford compound 11 in 86% yield (205 mg).

1H NMR (500 MHz, DMSO-d6) δ 8.48 (t, J=5.8 Hz, 8H, Tre-D-amides), 8.09-7.75 (m, 54H, Tre-D-core-amides+triazole H), 7.18 (s, 16H, D-Ar-H), 5.32 (s, sugar-H), 4.97 (s, sugar-H), 4.87-4.68 (m, sugar-H), 4.67-4.25 (m, sugar-H), 4.21-3.98 (m, PEG-H+sugar-H), 3.91-3.72 (m, PEG-H), 3.71-3.52 (m, PEG-H), 3.51-3.26 (m, PEG-H+core-H), 3.26-3.14 (m, core-H), 3.13-3.02 (m, core-H), 2.98 (t, J=9.3 Hz, Core-H), 2.82-2.52 (m, core-H), 2.34-2.02 (m, 42H, sugar-H), 1.84-1.75 (m, 16H, sugar-H). 1H NMR (500 MHz, D 2°) δ 7.89 (m, Tre-d-triazoles), 7.06 (s, 16H, Tre-D-Ar), 5.02 (t, J=3.3 Hz, sugar-H), 4.55 (m, PEG-H), 3.58 (m, PEG-H), 2.35 (m, sugar-H), 1.80 (m, sugar-H), and 1.22 (s, sugar-H). 13C NMR (125 MHz, DMSO-d6) δ 166.0, 152.2, 144.1, 140.3, 129.7, 124.9, 124.7, 122.4, 106.7, 93.7, 73.2, 73.1, 72.3, 71.9, 71.8, 70.5, 70.4, 70.3, 70.2, 70.1, 69.4, 69.2, 68.8, 63.8, 61.2, 51.2, 49.6, 49.1, and 29.5.

Synthesis of compound 12: To activate, 5-hexynoic acid (85 mg, 15.0 eq, 0.76 mmol) was dissolved in anhydrous DMF (10 mL) and treated with EDC·HCl (238.8 mg, 25.0 eq, 1.25 mmol). The reaction mixture was stirred for 10-15 minutes. Next, a solution of Trehalose dendrimer 11 (1 g, 1.0 eq, 0.05 mmol) in DMF (5 mL) was added dropwise to the stirred activated hexynoic acid solution. Following this, DMAP (43 mg, 7.0 eq, 0.35 mmol) was added to the reaction mixture. The entire reaction was allowed to stir at room temperature for 24 h. The reaction completion was confirmed via HPLC analysis by a shift in the retention time of the starting compound. The dialysis was performed using a 1 kDa dialysis membrane in DMF for 12 h. The final dialysis was performed against DI water. The product was lyophilized to afford compound 12 in 90% yield. 1H NMR (500 MHz, DMSO-d6) δ 8.48 (s, 8H, Tre-D-amides), 8.00-7.79 (m, 54H, Tre-D-core-amides+triazole H), 7.18 (s, 16H, D-Ar-H), 5.43-4.91 (m, sugar-H), 4.91-4.65 (m, sugar-H), 4.69-4.28 (m, sugar-H), 4.28-3.88 (m, PEG-H), 3.87-3.41 (m, PEG-H+core-H), 3.30-2.91 (m, PEG-H+core-H), 2.87-2.75 (m, core-H), 2.71-2.53 (m, sugar-H+core-H), 2.48-2.29 (m, sugar-H+linker-H), 2.28-2.06 (m, sugar-H+linker-H), 1.82-1.65 (m, 42H, sugar-H+linker-H).

Synthesis of Tre-D-Cy5 (13): To a stirred solution of compound 12 (25 mg, 1.0 eq, 0.0012 mmol) in 1 mL of DI water in a reaction vial, was added solution of Cy5 Azide (3.7 mg, 3.0 eq, 0.0035mmol) dissolved in 1 mL DMF, followed by the addition of CuSO4.5H2O (10 mol % per acetylene) dissolved in 0.1 mL DI water. After 2 minutes of stirring, sodium ascorbate (15 mol % per acetylene) was added, and the reaction was heated at 40 °C for 10 h. Upon completion, the dialysis was performed using a 1 kDa (MWCO) dialysis membrane in DI water for 12 h. The aqueous solution was lyophilized to afford compound 13 in 89% yield.

1H NMR (500 MHz, DMSO-d6) δ 8.40 (s, 2H, Cy5-Ar), 8.30 (s, 4H, Cy5-Ar), 7.91 (s, 20H, Tre-D amides and triazoles+Cy5-Ar), 7.86-7.69 (m, 35H, Tre-D-core-amides), 7.64-7.53 (m, 12H, Tre-D+Cy5-Ar), 7.42 (d, J=8.1, 10H, Tre-D+Cy5-Ar), 7.31-7.22 (m, 12H, Cy5), 7.11 (s, 16H, Tre-D-Ar), 6.55-6.48 (m, Cy5-H), 6.24 (d, J=13.9, Cy5), 5.24-5.23 (m, sugar-H), 4.89 (s, sugar-H), 4.78-4.67 (m, core+sugar-H), 4.57-4.37 (m, core+sugar-H), 4.29 (s, cor+sugar-H), 4.17-3.94 (m, sugar-H), 3.64 (d, J=46.8, PEG-H), 3.57-3.38 (m, PEG-H), 3.19-2.89 (m, core+sugar-H), 2.36 (s, core+Cy5-H), 2.11-1.85 (m, Cy5-H+linker-H), 1.62 (s, Cy5-H), 1.56-1.41 (m, linker-H), 1.32-1.00 (m, linker-H+Cy5-H).

Synthesis of Compound 16: A nitrogen-purged reactor vessel was charged with solution of (E)-6-iodo-3-(2-(pyridin-2-yl)vinyl)-1-(tetrahydro-2H-pyran-2-yl)-1H-indazole 14 (500 mg, 1.0 eq, 1.16 mmol), and K3PO4 (615 mg, 2.5 eq, 2.90 mmol) in DMF (2 mL). Pd(dppf)Cl2·CH2Cl2 (47 mg, 0.05 eq, 0.058 mmol) and 2-mercapto-N-methylbenzamide 15 (293 mg, 1.5 eq, 1.75 mmol) were added and the reaction mixture was purged with N2 for an additional 5 minutes. The reaction mixture was heated at 80 °C for 12 h. Once TLC indicated the reaction was complete, the reaction mixture was evaporated using rotary evaporator and purified on a silica column to afford compound 16 in 73 % yield.

1H NMR (500 MHz, DMSO-d6) δ 8.62 (d, J=4.3 Hz, 1H), 8.32 (d, J=8.2 Hz, 1H), 8.10 (s, 1H), 8.01-7.91 (m, 2H), 7.83 (t, J=7.2 Hz, 1H), 7.71 (d, J=7.8 Hz, 1H), 7.64 (d, J=16.3 Hz, 1H), 7.41 (t, J=7.5 Hz, 1H), 7.38-7.25 (m, 3H), 6.83 (d, J=8.1 Hz, 1H), 5.96 (dd, J=9.7, 2.4 Hz, 1H), 3.90 (s, 4H), 3.84-3.72 (m, 1H), 2.49-2.39 (m, 1H), 2.09-1.97 (m, 2H), 1.82 1.68 (m, 1H), 1.59 (s, 2H). 13C NMR (125 MHz, DMSO-d6) δ 166.52, 155.07, 150.09, 142.20, 142.00, 141.72, 137.42, 133.45, 131.22, 130.98, 130.90, 128.58, 127.98, 127.14, 125.60, 123.36, 123.23, 123.17, 122.97, 122.66, 117.88, 84.47, 67.16, 52.81, 29.38, 25.19, 22.61.

Synthesis of Compound 17: To a stirred solution of compound 16 (350 mg, 1.0 eq, 0.74 mmol) in MeOH: THF: H2O (1:1:1) (5 mL) was added NaOH (30 mg, 2.0 eq, 1.48 mmol). The reaction mixture was stirred at room temperature for 2 h. After confirming completion of the reaction by TLC, the reaction mixture was acidified with saturated citric acid solution, extracted with ethyl acetate (20 mL×3), dried over Na2SO4, and concentrated to obtain a crude product. The crude was purified n a silica column to afford compound 17 in 90 % yield.

1H NMR (500 MHz, DMSO-d6) δ 13.28 (s, 1H, −NH), 8.63 (d, J=4.2 Hz, 1H), 8.32 (d, J=8.4 Hz, 1H), 8.10 (s, 1H), 8.02-7.90 (m, 2H), 7.83 (t, J=7.6 Hz, 1H), 7.71 (d, J=7.8 Hz, 1H), 7.65 (d, J=16.3 Hz, 1H), 7.42-7.33 (m, 2H), 7.33-7.28 (m, 1H), 7.23 (t, J=7.5 Hz, 1H), 6.77 (d, J=8.1 Hz, 1H), 5.97 (d, J=10.1 Hz, 1H), 3.95-3.86 (m, 1H), 3.84-3.72 (m, 1H), 2.47 2.38 (m, 1H), 2.08-1.95 (m, 2H), 1.76 (tt, J=14.6, 6.7 Hz, 1H), 1.66-1.55 (m, 2H). 13C NMR (125 MHz, DMSO-d6) δ 167.9, 155.1, 150.1, 142.2, 142.2, 141.8, 137.4, 133.0, 131.4, 131.3, 130.9, 128.8, 127.9, 127.5, 125.3, 123.4, 123.2, 123.2, 122.9, 122.6, 117.9, 84.5, 67.2, 29.4, 25.2, 22.6.

Synthesis of Compound 18: The compounds 17 (250 mg, 1.0 eq, 0.55 mmol) and azido-PEG 6 amine (215 mg, 1.3 eq, 0.71 mmol) were dissolved in dry DMF (5 mL). DIPEA (0.2 mL, 1.0 eq, 0.74 mmol) and HATU (316 mg, 1.0 eq, 0.83 mmol) were added to the solution, and the mixture was stirred at room temperature for 1 h. The completion of the reaction was confirmed by TLC analysis. EtOAc (100 mL) was added, washed with water and brine, dried over Na2SO4. After filtration and evaporation of the solvent, the crude material was purified on a silica column to afford compound 18 in 54 % yield.

1H NMR (500 MHz, DMSO) δ 8.62 (d, J=5.3 Hz, 1H), 8.51 (t, J=5.7 Hz, 1H), 8.22 (d, J=8.5 Hz, 1H), 7.98-7.90 (m, 2H), 7.82 (t, J=7.6 Hz, 1H), 7.69 (d, J=7.8 Hz, 1H), 7.61 (d, J=16.3 Hz, 1H), 7.51 (d, J=7.5 Hz, 1H), 7.37-7.23 (m, 3H), 7.22 (d, J=8.5 Hz, 1H), 7.00 (d, J=7.9 Hz, 1H), 5.96-5.88 (m, 1H), 3.90 (d, J=11.3 Hz, 1H), 3.83-3.72 (m, 1H), 3.60-3.34 (m, 24H), 2.10-1.97 (m, 2H), 1.65-1.54 (m, 2H), 1.30-1.21 (m, 2H). 13C NMR (125 MHz, DMSO-d6) δ 167.9, 162.8, 162.1, 155.1, 150.1, 142.1, 141.6, 137.4, 136.7, 136.5, 133.3, 130.9, 130.7, 130.0, 128.4, 127.2, 126.4, 123.3, 123.2, 122.5, 122.1, 115.7, 84.5, 70.3, 70.3, 70.2, 70.2, 70.2, 70.1, 70.1, 70.0, 69.7, 69.3, 69.2, 67.1, 50.4, 44.6, 38.7, 36.3, 31.2, 29.4, 25.2, 22.6.

Synthesis of Compound 19: Compound 18 (200 mg, 1.0 eq, 0.29 mmol) was dissolved in trifluoroacetic acid (2 mL), and the reaction mixture was stirred at 60 °C for 1 h until completion. The completion of the reaction was monitored using TLC. Trifluoroacetic acid (TFA) was then evaporated under vacuum distillation. The saturated aqueous sodium bicarbonate solution was added dropwise to the reaction mixture until the pH reached 7-8. The resulting compound was extracted with ethyl acetate (3×20 mL), washed with brine, dried over anhydrous Na2SO4, and concentrated under reduced pressure to obtain the crude product. The crude product was purified on a silica column to afford compound 19 in 70% yield.

1H NMR (500 MHz, DMSO-d6) δ 13.47 (s, 1H), 8.70 (d, J=5.0 Hz, 1H), 8.56 (t, J=5.7 Hz, 1H), 8.26 (d, J=8.4 Hz, 1H), 8.06 (d, J=16.5 Hz, 1H), 7.98 (t, J=7.9 Hz, 1H), 7.86 (d, J=8.1 Hz, 1H), 7.66 (t, J=8.2 Hz, 2H), 7.56 (dd, J=7.5, 1.8 Hz, 1H), 7.47-7.32 (m, 3H), 7.26 (d, J=8.4 Hz, 1H), 7.13 (d, J=7.6 Hz, 1H), 3.87-3.33 (m, 24H).

Synthesis of Compound 20: To a stirred solution of compound 12 (150 mg, 1.0 eq, 0.008 mmol) in 1 mL DI water was added solution of compound 19 (56 mg, 13 eq, 0.09 mmol) dissolved in 1 mL DMF followed by the addition of CuSO4.5H2O (10 mol % per acetylene) dissolved in 1mL DI water. After 2 minutes of stirring, sodium ascorbate (15 mol % per acetylene) was added, and the reaction mixture was stirred at 40 °C for 16 h. Upon completion, the compound was purified on TFF using a 3 kDa dialysis membrane. The aqueous solution was lyophilized to afford compound 20 in 90% yield. HPLC purity: 99.7%, retention time: 11.02 minutes.

1H NMR (500 MHz, DMSO-d6) δ 13.29 (s, 10H, Axi-NH), 8.53 (d, J=5.0 Hz, 10H, Axi-H), 8.47-8.37 (m, 18H, Axi-H), 8.13 (d, J=8.5 Hz, 10H, Axi-H), 7.97-7.80 (m, 45H, Triazoles+amides+Axi-H), 7.80-7.64 (m, 33H, Triazoles+amides), 7.59 (d, J=7.9 Hz, 10H, Axi-H), 7.55-7.47 (m, 17H, Axi-H), 7.45-7.37 (m, 12H, Axi-H), 7.29-7.15 (m, 30H, Axi-H), 7.15 7.05 (m, 26H, Axi-H+D-Ar-H), 6.98 (d, J=7.8 Hz, 10H, Axi-H), 5.17-4.86 (m, sugar-H), 4.85-4.65 (m, Sugar-H), 4.62-4.26 (m, sugar-H), 4.22-3.90 (m, sugar-H+PEG-H), 3.89-3.48 (m, sugar-H+PEG-H), 3.41-3.18 (m, PEG-H), 3.17-2.86 (m, PEG-H), 2.77-2.62 (m, core-H), 2.62-2.44 (m, core-H) , 2.29 (s, 41H, core-H+linker-H), 2.16-1.93 (m, 42H, core-H+linker-H), 1.84-1.66 (m, core-H+linker-H, 42H). 13C NMR (125 MHz, DMSO-d6) δ 168.0, 166.1, 155.3, 152.2, 150.0, 144.1, 140.3, 137.4, 135.9, 133.1, 130.8, 130.6, 129.7, 128.4, 126.7, 125.9, 124.8, 123.1, 123.0, 122.2, 120.7, 106.7, 97.2, 93.7, 79.7, 79.4, 79.2, 73.3, 73.1, 73.0, 72.3, 71.9, 71.8, 70.5, 70.4, 70.3, 70.2, 70.1, 70.0, 69.5, 69.4, 69.2, 68.8, 63.8, 61.2, 51.2, 49.7, 38.8, 33.3, 25.6, 24.8, 21.9.

Synthesis of Tre-D-Axi-Cy5 (compound 21): To a stirred solution of compound 20 (50 mg, 1.0 eq, 0.0012 mmol) in 1 mL DI water in a microwave vial was added the solution of Cy5 azide (4.3 mg, 3.5 eq, 0.0042 mmol) dissolved in 1 mL DMF followed by the addition of CuSO4.5H2O (10 mol % per acetylene) dissolved in 0.1 mL DI water. After 2 minutes of stirring, sodium ascorbate (15 mol % per acetylene) was added, and the reaction was conducted under microwave irradiation at 40 °C for 10 h. Upon completion, the mixture was subjected to dialysis using a 1 kDa dialysis membrane in DI water for 12 hours. The resulting aqueous solution was lyophilized to afford compound 21 in 88% yield. 1H NMR (500 MHz, DMSO-d6) δ 8.70-8.36 (m, 26H, Axi-H+Cy5-H), 8.36-8.01 (m, 25H, Axi-H), 8.03-7.69 (m, 83H, Tre-D-triazoles+Axi-H), 7.67-7.33 (m, 32H, Tre-D-triazoles+amides), 7.33-7.18 (m, 21H, Axi-Ar+Tre-D-triazoles), 7.18-7.11 (m, 17H, Axi-Ar), 7.11 (s, 16H, Tre-D-Ar), 7.03-6.89 (m, 10H, Axi-Ar), 6.50 (s, 2H, Cy5-H), 6.24 (d, J=13.4, 4H, Cy5-H), 5.48-4.68 (m, sugar-H+core-H), 4.59-4.28 (m, PEG+sugar-H), 4.23-3.94 (m, PEG), 3.84 3.40 (m, PEG-H), 3.18-2.81 (m, PEG+sugar-H+core-H), 2.63-2.50 (m, core+linker-H), 2.35-1.91 (m, core+Cy5-H+linker-H), 1.88-1.59 (m, Cy5-H+linker-H, core-H), 1.55-1.41 (m, Cy5-H+linker-H), 1.27-0.99 (m, Cy5-H+linker-H).

### Stability Studies

### Tre-d-Axitinib Formulation Shelf-Stability Studies

Tre-D-Axitinib was formulated at 50 mg/mL in PBS and sterile filtered through 0.4 μm filters. The formulations were kept at room temperature and 4 °C. The aliquots were analyzed via HPLC for the purity at 0 h, 1 day, 2 day, 3 day, 7 day, 15 day, and 28 day time points.

### Tre-D-Axitinib Formulation Stability Studies at Physiological Conditions

In vitro drug release studies were performed under two conditions: plasma conditions using phosphate-buffered saline (PBS) at pH 7.4, and intracellular conditions using citrate buffer at pH 5.5, supplemented with esterase at 37° in an incubator. The Tre-D-Axi conjugate was dissolved at a concentration of 2 mg/mL in each respective buffer and underwent incubation at 37 °C with continuous shaking to replicate the physiological conditions. At predetermined time intervals, samples were withdrawn, promptly quenched with an equivalent volume of methanol, and stored at −20 °C until further analysis. The released drug was quantified using high-performance liquid chromatography (HPLC), by comparing the results to a standard curve established for the free drug, Axitinib, on the HPLC system.

### In Vitro Studies-Experimental Section

### Materials and Reagents

Phosphate buffer saline (PBS), 4′,6-diamidino-2-phenylindole (DAPI), and 3-(4,5-dimethylthiazol-2-yl)-2,5 diphenyltetrazolium bromide (MTT), were procured from Aaron Chemicals. Dulbecco's Modified Eagle Medium (DMEM) was purchased from Cytiva, Fetal bovine serum (FBS) was obtained from Gibco Scientific. All the cell lines were procured from American Type Culture Collection (ATCC). Angiogenesis and BrdU cell proliferation assay kit was purchased from Abcam. All these above-mentioned reagents were used as such.

### Cell Culture

Human Retinal Microvascular Endothelial Cells (HRMVECs; #ACBRI 181), were obtained from Cell Systems, Kirkland, WA. We tested these cells for their EC authenticity by immunostaining them for CD31 and vWF. The HRMVECs were regularly checked for mycoplasma contamination. These HRMVECs were grown in EGM2 medium with amphotericin B (0.25 μg/mL) and gentamycin (10 μg/ml) and kept at 37 °C in a humidified incubator (95% air and 5% CO2). The cells were synchronized in a serum-free media for approximately 24 h to achieve quiescent state.

### Cell Viability Assay

In order to evaluate the efficacy of the Tre-D-Axitinib conjugate under in vitro/in vivo settings, we performed primary cellular compatibility (3-(4,5-dimethylthiazol-2-yl)-2,5-diphenyltetrazolium bromide) (MTT) assay on two cell lines, Human umbilical vein endothelial cells (HUVECs) and macrophages (RAW-Blue) following previous reports. For this, already grown cells were seeded in 96 well plate (104 cells/well) and grown overnight in CO 2 incubator. The media was aspirated and fresh media along with the samples at different concentrations (1, 5, 25 and 50 μM) were treated with the corresponding cells for 48 h at 37 °C. The media containing samples were again aspirated and the cells were washed with 1× PBS thoroughly. Following this, the cells were incubated with 10 μl MTT (5 mg/mL) for ~3 h. The MTT was aspirated carefully and ~150 μL of culture grade DMSO was added to each well to dissolve the formazan crystals. Finally, a UV-vis based read-out was taken at 570 nm to evaluate the cellular viability using BioTek, synergy multi-mode microplate reader (Thermo Scientific Multiskan® SkyHigh Microplate Spectrophotometer). All the samples were taken in triplicates with proper controls.

### Angiogenesis Assay

For evaluating the anti-angiogenesis efficacy of the Tre-D-Axitinib conjugate, an angiogenesis assay was performed in vitro according to the kit protocol using the Angiogenesis Assay Kit (ab204726). Briefly almost 50 μL of the provided kit matrix (ECM) was added to the each of the black colored clear bottom 96 well-plate for 1 h at 37 °C in a CO2 incubator, till it gets solidified. The cultured HUVEC cells were seeded into these wells at a density of 1×105 cells/well and grown overnight at 37 °C. Next day, these cells were incubated with the corresponding Tre-D-Axitinib and free Axitinib at different concentrations (1, 5, 25 and 50 μM), and dendrimer control (Tre-D, 50 μM) for 12 h at 37 °C along with the control wells with added matrix. The media was carefully aspirated without disrupting the matrix and washed with the provided elution buffer. Finally, the cells were stained with the staining dye provided with the kit for ~30 min at 37 °C and imaging was carried out using inverted fluorescence microscope (Zeiss Axiovert® fluorescence microscope at 5× magnification). The images were finally taken by a digital camera attached to the microscope by zooming out to get the image of the whole wells. After the imaging was done, the images were analyzed using Carpentier et al.'s (2020) angiogenesis plug for ImageJ software (NIH, v1.54j). The interaction between cells was expedited using ImageJ-programmed macro language for visualizing the micrographs. The plug-in automatically identified and analyzed different angiogenic parameters including tube length, junctions, branches, meshes, segments and extremities for determining the angiogenic capacities of corresponding dendrimers onto the HUVEC cells. All the measurements were performed in triplicates with proper controls.

### Cell Proliferation Assay

For evaluating the effect of dendrimers on the cell proliferation of the HUVEC cells, a kit-based BrdU cell proliferation assay was performed. For this, almost 2×105 cells were seeded in a 96-well plate and put in a CO2 incubator for growth (37 °C, 5% CO2). Following this, the HUVECs were treated with the Tre-D-Axitinib conjugate at different concentrations (1, 5, 25 and 50 μM) along with the same concentration of Axitinib, and the highest concentration of Tre-D control (50 μM) for 12 h. Approximately 20 μL of BrdU reagent was added to all the sample wells and incubation was done for 25 min in CO2 incubator. The sample solution was carefully aspirated, and wells were washed properly using plate wash buffer. The cells were fixed using the fixing solution provided in the kit for 20 min followed by washing again. For each of the sample wells, 100 μL anti-BrdU detector antibody was added, and incubation was done for 1 h at 37 °C followed by washing and gentle drying afterwards. After this, 100 μL/well goat anti-mouse IgG peroxidase antibody was added to the wells. Further, all the solutions were aspirated, and the wells were washed several times and dried properly. To each well, 100 μL of chemiluminescent substrate was applied and incubated for ~10 min at room temperature in the dark. The luminescence reading was immediately taken using a Synergy H1 hybrid multi-mode microplate reader (BioTek Instruments). All the measurements were carried out in triplicates; with proper controls, blank (media alone) and background (cells only, no BrdU reagent).

The HRMVECs proliferation was assessed using BD Pharmingen™ BrdU flow kit (#559619, BD Biosciences, San Jose, CA) following the manufacturer's standard protocol. HRMVECs were cultured in 60-mm dishes (2×106 cells/mL) until they reached confluence. BrdU (10 μL/mL, 1 mM solution) was then added and incubated for 2 h at 37 °C. To fix and permeabilize the cells, BD Cytofix/Cytoperm buffer was used, and the cells were left at room temperature (RT) for 30 minutes. Following the removal of the fixative, the cells were meticulously washed using BD Perm/Wash buffer. The cells were subsequently permeabilized using BD Cytoperm® Plus buffer and incubated on ice for 10 minutes. After another washing cycle, the cells were fixed using BD Cytofix/Cytoperm® solution for 5 minutes at room temperature. Following fixation, the cells were exposed to DNase solution for one hour at 37 °C. Thereafter, the cells were washed and incubated for 20 minutes at room temperature with 50 μL of a working solution of FITC-conjugated anti-BrdU antibody. Following a further wash, 20 μL of 7-AAD solution was introduced. The cells were ultimately resuspended in 1 mL of staining buffer. The specimens were examined on a BD LSRII flow cytometer.

### Cell Migration Assay

The effect of Tre-D-Axitinib and free drug on the migration of HRMVECs and HUVECs in presence or absence of VEGFA were carried out using scratch/cell migration assay following the published reports with slight modifications. In brief, 2×105 cells were seeded in a 12 well plate and grown overnight at 37 °C in a CO2 incubator. The media was aspirated, cells were washed using 1× PBS and a clean scratch was made using 200 μL micropipette tips manually followed by washing again to remove the scratched cells. Fresh media (supplemented with ~1% FBS) was added to the cells along with the dendrimers at corresponding concentrations (1, 5, 25 and 50 μM) were incubated with this media. Light microscopy imaging was performed at 0, 24 and 48 h intervals to see the changes in the migration behavior under the influence of dendrimers, using Zeiss Axiovert® 135 TV microscope or EVOS® M5000 Imaging System at 4× magnification (Thermo Fisher Scientific, Waltham, MA). The anti-migration efficacy of the dendrimers was evaluated using ImageJ (NIH, v1.54j) plugin with high throughput image analysis of in vitro scratch. Samples triplicates were taken with proper controls. The percentage of wound closure was determined using the formula: [(total wound area at 0 hours)−wound area (24 hours)/total wound area at 0 hours]×100.

### Spheroid Sprouting Assay

We subjected the HRMVECs to a three-dimensional spheroid sprouting assay, following the protocols laid out by Bisen et al. Briefly, the HRMVECs were first labeled with BCECF-AM (10 μM). To make spheroids (about 400 cells/spheroids), 80,000 cells were suspended in 4 mL of EBM 2 media supplemented with 20% methocel®. We placed the 25 mL cell suspension drops on a 150 mm cell culture plate and incubated them in a humidified 5% CO2 incubator at 37 °C for 24 hours to form spheroids. Next, we washed the spheroids with PBS and stored them in two milliliters of ice-cold methocel® that contained 10% FBS. Thereafter, 4 ml of a collagen solution (1 mg/ml of collagen in EBM 2 media) was mixed with the spheroid solution, and 250 mL of the resulted solution was pipetted in a 24-well cell culture plate and incubated for one hour at 37 °C in a humidified CO2 incubator to solidify/polymerize the collagen. After an hour, 250 mL of EGM 2 medium was added to each well of the 24-well plate that had the spheroids implanted. The plate was then kept in a 5% CO2 incubator at 37 °C for 48 hours. We used a Zeiss LSM 800 microscope to capture the spheroid images after 48 hours.

### Tube Formation Assay

Growth factor-reduced Matrigel® (35420, Corning Life Sciences, Corning, NY) was used to perform the HRMVECs tube formation experiment. We transfected control and specific siRNAs into HRMVECs, quiesced, suspended them, and then added them to a 24-well Matrigel®-coated culture dish. The cells were incubated at 37 °C for 6 hours. We analyzed tube formation using the EVOS® M5000 microscope (Thermo Fisher Scientific, Waltham, MA). Tube length in HRMVECs was measured using NIH ImageJ version 1.43 software and reported in micrometers.

### Cell Uptake Studies

For evaluating the mechanism of Tre-D and Tre-D-Axitinib uptake by HUVECs, a cell uptake study was carried out in presence of cell trafficking inhibitors, including cytochalasin B (GLUT8 inhibitor), chlorpromazine (CPZ) and methyl β-cyclodextrin (MβCD) for clathrin-and caveolae-mediated endocytosis inhibition, and Amiloride (GLUTs) inhibitor. We followed our previous published protocols with some modifications.

### In Vivo Studies—Experimental Section

### Experimental Animals

We purchased C57BL/6J mice from Jackson Laboratory located in Bar Harbor, ME, USA. These mice were maintained and bred in the Wayne State University DLAR animal facility located in Detroit, MI, USA. We provided the mice with unrestricted access to food and water and were kept in a 12-hour light and 12-hour dark cycle setting. This study included both female and male mouse pups ranging from postnatal day 12 (P12) to postnatal day 17 (P17). All animal experimental procedures were approved by Institutional Animal Care and Use Committee (IACUC-23-07-5936) of Wayne State University, and this study has been conducted in strict accordance with the Guide for the Care and Use of laboratory Animals as revised by the National Institutes of Health (NIH).

### Sex As a Biological Variable

In our study, we used both male and female mice in our OIR studies. We have not observed any differences in both male and female mouse pups, and thus sex was not considered a biological variable.

### Intraperitoneal Injections

Mice pups were intraperitoneally administered with 50 microliters of Tre-D, or Tre-D-Cy5, or Tre-D-Axitinib or Tre-D-Axitinib-Cy5 at P12 using a 30 G needle.

### Immunofluorescence Staining

Following hyperoxia exposure, mice pups were administered with Tre-D-Cy5 at P12. At P17, the pups were euthanized, eyes enucleated, and embedded in optimal cutting temperature (OCT) compound. 10-μm cryosections were made from the retina central part. To know the location of rhodamine labeled-nontargeted siRNA in the retina, the sections were probed with rat anti-mouse CD31(1:100) primary antibodies and Alexa Fluor® 488-conjugated secondary antibodies. These sections were then examined under Zeiss LSM800 confocal microscope and images were captured using image analysis software Zen (Carl Zeiss Imaging Solutions GmbH).

### Oxygen-Induced Retinopathy (OIR)

At P7, mice pups along with dams were kept in BioSpherix® chamber for five days and exposed to 75% oxygen (P7 to P12), and then returned to room air. The mice littermates of the same age continued at room air (21% oxygen), were considered as controls. At P17, the pups were euthanized, their eyes enucleated and fixed. The retinas were isolated and stained with rhodamine labelled isolectin B4. After staining, the retinas were flat mounted and observed under a Zeiss LSM 800 confocal microscope and retinal neovascularization was quantified using Nikon NIS-Elements advanced research software. Retinal neovascularization was highlighted in red, and the retinal neovascularization was calculated as fluorescence intensity of the highlighted area/total fluorescence intensity of the retina×100. The percent avascular area was defined as total avascular area/total retina area×100.

### Statistics

We performed all experiments at least three times. The data are displayed as mean±SD in the graphs. The normality of the data distribution was assessed using the Kolmogorov-Smirnov test with Lilliefors' adjustment prior to conducting a parametric test. We used unpaired t-tests to assess differences between two groups and one-way ANOVA with Bonferroni's correction for trials involving multiple groups. Statistical analyses were conducted using GraphPad Prism® 9, with a significance threshold set at P<0.05.

### Ex Vivo Dendrimer Quantification

The frozen organs (heart, lungs, liver, kidneys, spleen, and brain) were thawed gradually on ice and weighed. Tissue samples were dissected, and specific amounts from each organ were collected. The tissues were homogenized in methanol at a ratio of 1 mL per 100 μg of tissue using stainless steel beads and a tissue homogenizer. After homogenization, the samples were centrifuged at 4 °C, and the clear supernatant was transferred to protein Eppendorf LoBind® tubes for storage at −80 °C. For fluorescence quantification, the thawed supernatants were centrifuged again, and fluorescence intensity was measured using a Fluoromax® spectrofluorophotometer. Cy5 fluorescence (λex=645 nm, λem=662 nm) was recorded and corrected for background fluorescence from control tissues. The fluorescence values were then converted to Tre-D-Axitinib-Cy5 concentrations using calibration curves at different slit widths.

### Results and Discussion

In this study, we aim to design an innovative dendrimer-based smart, systemic, and minimally invasive delivery system that can precisely deliver anti-VEGFA therapies to diseased NV areas in retina. This approach seeks to replace current painful regimen of intraocular injections of anti VEGF drugs, which are the first-line treatment for retinal NV. The complexity in the design of nanomedicines is a major bottleneck for their large-scale production and scalability and often lead to failure during clinical development. Therefore, it is crucial to rationally design disease-directed nanotherapies with simplified yet efficient synthetic approaches to ensure their rapid translation from bench to bedside.

### Rationale for the Development of Tre-D Technology

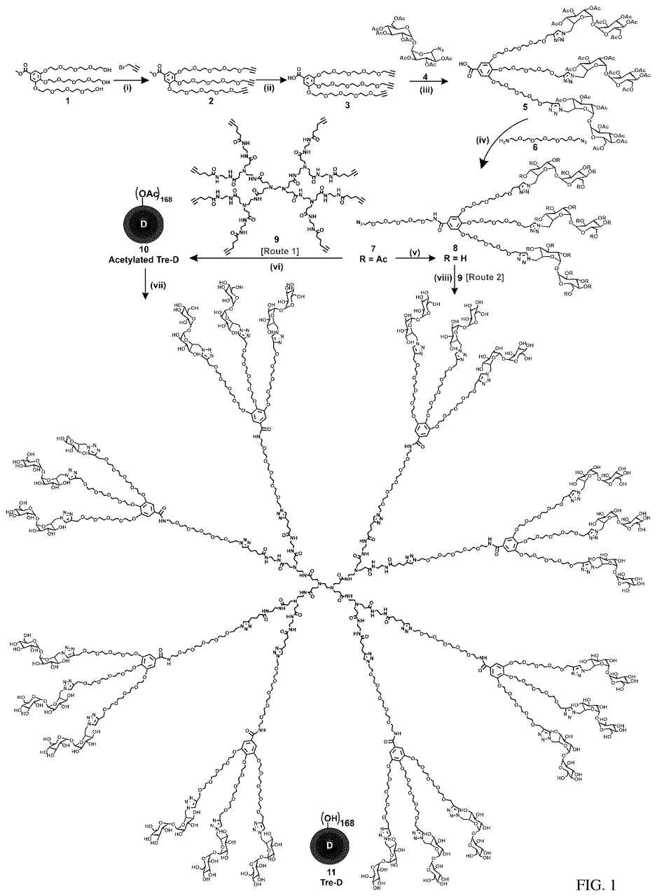

Chemically, the Tre-D is composed of three distinct layers at each generation (FIG. 1). Tre-D is a generation 3 dendrimer where the generation 1 polyamidoamine (PAMAM) dendrimer serves as the core, then comes the second generation which is composed of biocompatible gallic acid building blocks, and finally a disaccharide of glucose, trehalose molecules, complete the 3rd layer of Tre-D platform. We used trehalose as the peripheral layer of Tre-D since trehalose is a biocompatible, FDA approved material which is used in pharmaceuticals as a stabilizing agent due to its ability to stabilize biological molecules and proteins.

Tre-D is constructed in fewer reaction steps, maintaining the orthogonality, enabling the rapid formation of highly dense flawless structures (FIG. 1). In contrast, traditional dendrimer synthesis methods for producing commercially available dendrimers, are often time-consuming, labor intensive, low yielding and synthetically complex. For example, the synthesis of a widely used fourth-generation PAMAM dendrimer typically requires 6 to 8 weeks of meticulous work, involving 8 to 10 reaction steps to generate 64 hydroxyl groups at the surface. To overcome these challenges, we developed an expedited convergent synthetic methodology, incorporating mixed layers of building blocks at each dendrimer generation. This design features an 8-armed core and a dendron with 21 hydroxyl groups, seamlessly connected using highly efficient copper-catalyzed click chemistry (CuAAC). We designed the Tre-D around biocompatible building blocks and generally recognized as safe (GRAS) reagents to reduce the risk of any adverse effects. The dendrimer is composed of ~38wt % trehalose, ~36wt % polyethylene glycol (PEG), ~10wt % triazole, ~10wt % core, and ~6wt % gallic acid.

The developed procedure for dendrimer synthesis is significantly faster and more efficient than existing conventional methods, along with the reduced use of hazardous materials. This streamlined approach aligns with sustainability goals, promoting safer and more eco-friendly practices in the manufacturing of pharmaceuticals. The functionalization of the dendrimer surface with trehalose, a highly water-soluble disaccharide, significantly enhanced the solubility of Tre-D to approximately 800 mg/mL in aqueous solutions. Apart from bringing hydrophilicity in the molecule, the resulting Tre-D carries 168 hydroxyl groups on the periphery of the dendrimer which can be modified by variety of chemistries for the attachment of drugs and therapeutics, biomolecules, proteins, antibodies, or genes. Moreover, we use stable chemical linkages in the dendrimer backbone to avoid degradation under physiological conditions and provide intact clearance of the dendrimer (~5 nm) from off-target organs.

### Synthesis and Characterization of Tre-D

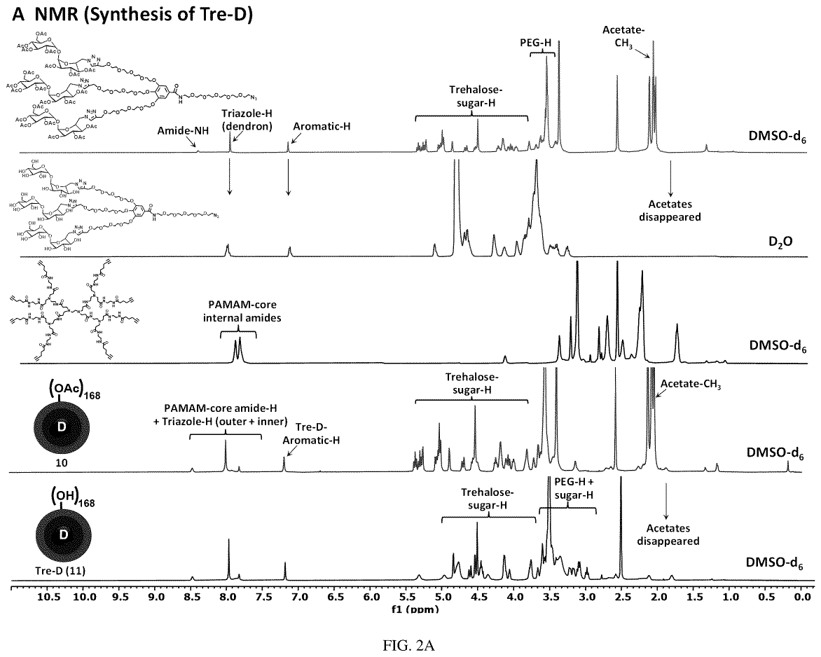

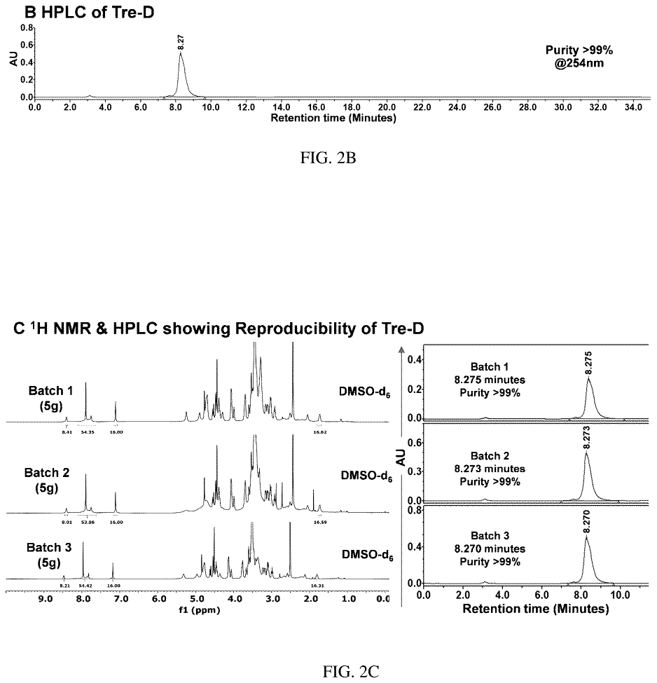

The synthesis pathway towards Tre-D started with the preparation of a clickable dendron 8, as depicted in FIG. 1. The process initiated with the propargylation of tri-hydroxyl gallate (1) using sodium hydride in anhydrous DMF, yielding compound 2. The successful synthesis of compound 2 was confirmed by 1H NMR, which exhibited a characteristic alkyne peak (3H) at δ2.43 ppm. In the next step, the hydrolysis of the methyl ester 2 afforded compound 3 in 78% yield. Next, the CuAAC reaction between tri-propargyl gallic acid 3 and peracetylated trehalose azide 4 afforded compound 5. The successful completion of the click reaction was confirmed by the disappearance of the alkyne proton signal at δ 2.31 ppm and the emergence of a characteristic triazole proton resonance (3H) at δ 8.00 ppm in the ¹H NMR spectrum. Additionally, the 1H NMR spectrum showed distinct acetate protons (63H) corresponding to the three trehalose sugars, appearing at δ 1.95-2.05 ppm. Subsequently, the EDC coupling between carboxylic acid 5 and azido-PEG-5-amine 6 afforded dendron 7 in 85% yield. The successful formation of compound 7 was confirmed by the presence of a characteristic amide proton at δ 8.46 ppm, along with signals from the OEG protons in the 1H NMR spectrum (FIGS. 1 and 2A). De-O-acetylation of dendron 7 was carried out under Zemplén transesterification conditions using sodium methoxide in methanol, yielding the fully deprotected trehalose dendron 8 in 92% yield. The successful completion of the reaction was verified by the disappearance of acetate protons in the δ 1.95-2.04 ppm region (FIGS. 1 and 2A). Next, we synthesized the 8-armed alkyne-functionalized G-1 PAMAM core (9) using our previously published procedure.

The characterization of dendrimers poses a significant challenge in synthetic chemistry due to their complex, highly branched structures and presence of huge number of protons which often produce dense and overlapping signals in 1H NMR spectra. Identifying key peaks in spectroscopic analysis is crucial for verifying the structural integrity and composition of the synthesized macromolecule. For ease of characterization, we synthesized Tre-D using two synthetic routes. We first opted to perform the reaction using acetate-protected trehalose azide dendron (7) (Route 1, FIG. 1). In the protected synthetic route, the octa-alkyne G-1 PAMAM dendrimer core (9) and the acetate-protected trehalose-azide dendron (7) was clicked together using CuAAC reaction. Conventional CuAAC conditions were employed where a catalytic amount of CuSO4·5H2O (5 mol % per alkyne) and sodium ascorbate (10 mol % per alkyne) was used, and the reaction was conducted under microwave (MW) irradiation at 40 °C for 15 h. This method yielded acetate-protected Tre-D (10) in 90% yield. The successful formation and the structure of the product were confirmed by the proton NMR showing the presence of triazole protons at δ 7.93 ppm and acetate protons at δ 1.82-2.00 ppm, corresponding to the attachment of 24 trehalose sugar units (FIGS. 1, 2A). This approach was particularly advantageous in characterizing the dendrimer, as the huge peaks from acetate protons occupy a region in the 1H NMR spectrum that avoids signal interference. The acetates were removed under Zemplén transesterification conditions using NaOMe in methanol, yielding the final G2 Tre-D (11) with 168 hydroxyl terminal groups in 90% yield. The complete deacetylation was confirmed by the ¹H NMR spectrum, which showed the disappearance of acetate proton signals, indicating the formation of the fully deprotected product (FIGS. 1, 2A). For route 2, we performed the CuAAC reaction between the core (9) and the deprotected trehalose-azide dendron (8) under similar conditions (Route 2, FIG. 1), leading to the formation of the final Tre-D (11). Both synthetic routes using protected and deprotected dendrons consistently produced excellent and reproducible results. Purification of the final Tre-D was accomplished through dialysis using a 3.5 kDa membrane. HPLC analysis of the dendrimer (11) confirmed purity exceeding 99% (FIG. 2B). The characterization of Tre-D and its intermediates was conducted using a range of techniques, including 1H NMR, 13C NMR, HPLC, MALDI-TOF, and HRMS. As shown in FIG. 2A, the 1H NMR spectra display the appearance and disappearance of characteristic peaks corresponding to various intermediates, which simplify the interpretation of the dendrimer's structure. Tre-D demonstrates exceptional water solubility (~800 mg/mL), which makes it a promising nanocarrier for hydrophobic drugs, hence eliminating the need for additional excipients for final drug products.

One of the primary challenges in translating nano-therapeutics into clinical applications is developing synthesis processes that are both reproducible and scalable. To overcome the batch-to-batch related heterogeneity associated with macromolecular synthesis, we kept the design of Tre-D very simple and straightforward, and stitched the building blocks together with highly facile and efficient chemical transformation, CuAAC. To evaluate the reproducibility of Tre-D synthesis, we produced several 5-gram-scale batches of Tre-D and compared their 1H NMR spectra along with purity via HPLC analysis. The 1H NMR spectra from three independent batches (FIG. 2C) consistently confirmed the presence of 24 trehalose dendrons on the surface, indicating the formation of defect-free dendrimer. Additionally, the HPLC chromatogram of each batch revealed peaks at the same retention time (8.2 minutes), further confirming batch-to-batch purity (FIG. 2C). The high and consistent yields across batches confirm the robustness of this synthetic method, suggesting its scalability for potential clinical translation.

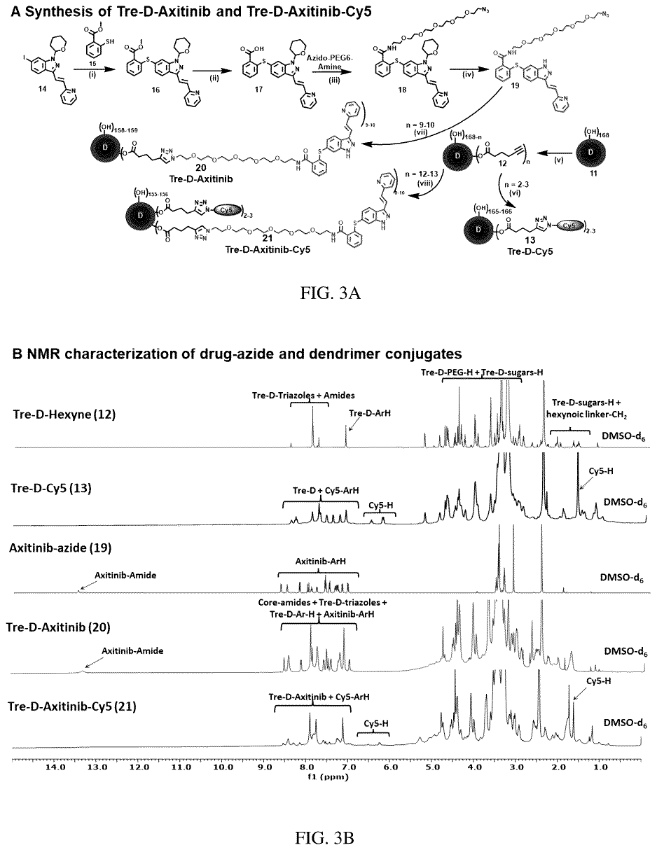

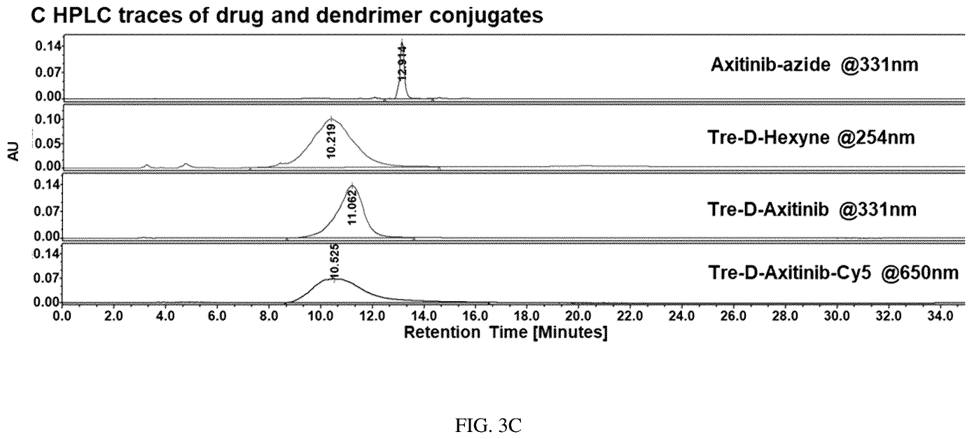

For in vitro and in vivo assessment of cellular uptake and organ biodistribution, Tre-D was surface labeled with the near-infrared fluorescent dye cyanine 5 (Cy5). The attachment of the Cy5 tag was achieved by introducing 3-4 linker arms of 5-hexynoic acid through an esterification reaction with the hydroxyl groups at the periphery of Tre-D. This reaction utilized EDC as a coupling reagent, resulting in the formation of compound 12 (FIG. 3A). The successful introduction of the 5-hexynoic linker was confirmed via 1H NMR analysis, which revealed additional methylene protons in the aliphatic region of the dendrimer. HPLC analysis further demonstrated a change in retention time, shifting from 8.2 minutes for Tre-D to 10.2 minutes for the modified Tre-D-Hexyne (12), confirming the successful attachment. In the next step, the acetylene-functionalized Tre-D-Hexyne (12) was conjugated with azide-terminated Cy5 using a CuAAC click reaction, yielding the fluorescently labeled Tre-D-Cy5 (13; FIG. 3A).

The successful conjugation of Cy5 to Tre-D was verified by the appearance of characteristic Cy5 proton peaks in the 1H NMR spectrum (FIG. 3B). Quantitative estimation of the number of Cy5 molecules linked to each dendrimer molecule was performed by proton integration, indicating the successful attachment of approximately 2-3 Cy5 molecules (FIG. 3B). Following conjugation, Tre-D-Cy5 exhibited an HPLC chromatogram at 650 nm, corresponding to the absorption wavelength of Cy5, with a purity greater than 99%. Additionally, the HPLC retention time shifted from 10.2 minutes to 9.4 minutes upon Cy5 conjugation.

### Synthesis and Characterization of Trehalose Dendrimer-axitinib (Tre-Dd-Axitinib) Conjugate

Next, we synthesized Tre-D-Axitinib, a conjugate of Tre-D with Axitnib. Axitinib is a highly potent TKI, known for its selective inhibition of VEGFR 1, 2, and 3, as well as other kinases such as c-KIT and PDGFRα/β, which are implicated in angiogenesis. It has demonstrated the inhibition of angiogenesis in both in vitro and in vivo models related to eye diseases. Axitinib is investigated as administered via intraocular routes for treating eye-related disorders due to its inability to cross the blood-retinal barrier. Moreover, its clinical use is hampered by poor water solubility (0.2 μg/mL) and side-effects. By using Tre-D dendrimers for targeted delivery of Axitinib to aberrant neovascular tufts, we aim to develop non-invasive therapies for retinal disorders.

To synthesize the Tre-D-Axitinib (Tre-D-Axitinib) conjugate (20), we initiated by partially modifying the hydroxyl groups of the Tre-D through esterification with 5-hexynoic acid (FIG. 3A). This reaction introduced approximately 12 acetylene arms (12) into the dendrimer structure which was confirmed via 1H NMR and HPLC. For the subsequent conjugation of Axitinib to Tre-D, we needed a clickable linker (preferably having terminal azide functionality) to Axitinib, which posed a challenge due to the lack of functional handle on Axitinib. An in-depth analysis of Axitinib's structure-activity relationship reveals that the pyrazole core, which interacts with the hinge region of the VEGFR2 domain, plays a crucial role in its anti-angiogenic activity. Literature reports indicate that modification at the methyl amide portion of the molecule is well-tolerated and maintains Axitinib's anti-angiogenic properties. Thus, we strategically modified the methyl amide bond on the phenyl ring of Axitinib through amide coupling with Amino-PEG5-azide, preserving the amide bond's interaction with VEGFR2 while adding an azide terminating linker. This is worth noting here that the conjugation of Axitinib in Tre-D-Axitinib conjugate is through non-cleavable amide bond and the Tre-D-Axitinib conjugate is designed as an anti-VEGF therapy.

The synthesis of the azide-terminated Axitinib analogue (compound 19) is depicted in FIG. 3A. To introduce an azide linker via an amide bond into the Axitinib structure, the synthesis began with the palladium-catalyzed cross-coupling of commercially available aryl iodide compound 14 with methyl-2-mercaptobenzoate 15 using Pd(dppf)Cl2′DCM in DMF, affording methyl benzoate 16 in 73% yield. The structure of compound 16 was confirmed by the presence of methyl ester protons at δ 3.89 ppm in the 1H NMR spectrum and the expected mass peak. Hydrolysis of ester 16 with sodium hydroxide yielded the free carboxylic acid 17. EDC-mediated coupling of 17 with azido-PEG5-amine afforded compound 18 in 76% yield, as confirmed by the disappearance of the methyl ester signal and the appearance of OEG protons in the 1H NMR spectrum. Finally, deprotection of the tetrahydropyran group on the pyrazole using trifluoroacetic acid afforded compound 19 in 70% yield. The structure of compound 19 was validated by the disappearance of the THP signals and the appearance of the NH proton in the pyrazole ring at δ 13.35 ppm, along with OEG protons in the range of δ 3.40-3.75 ppm (FIG. 3B).

After synthesizing azido-PEG5-Axitinib (19), we carried out its click reaction with Tre-D-hexyne (12) using previously established CuAAC conditions, successfully obtaining the Tre-D-Axitinib (20) conjugate (FIG. 3A). Purification was performed by dialysis using a 3 kDa membrane, first in 20% DMF/H2O to remove small-molecule impurities, followed by final dialysis in water, affording Tre-D-Axitinib in 86% yield. The characterization of Tre-D-Axitinib (20) conjugate was performed using proton integration method, with the sugar protons of Tre-D-Hexyne 12 (n=~12), observed at δ 1.71-1.80 ppm, serving as a reference (FIG. 3B). This analysis indicated that approximately ~9 Axitinib molecules were conjugated to each dendrimer, as confirmed by ¹H NMR spectroscopy (FIG. 3B). Additionally, we evaluated the purity of Tre-D-Axitinib through HPLC, which demonstrated a purity level exceeding 99% (FIG. 3C). There was a clear shift in the retention time upon conjugation of Axitnib-azide (12.9 minutes) to Tre-D-Hexyne (10.2 minutes) to afford Tre-D-Axitinib (11.0 minutes). We next attached a fluorescent tag Cy5 to Tre-D-Axitinib to investigate the cellular uptake mechanisms. The synthesis of Tre-D-Axitinib-Cy5 (21) was accomplished via a click reaction between Cy5 azide and the alkyne groups on the Tre-D-Axitinib conjugate (20). The confirmation of Cy5 attachment was performed through 1H NMR analysis, which revealed the presence of Cy5 protons, indicating that approximately two Cy5 molecules were linked to the dendrimer surface (FIG. 3B). HPLC analysis demonstrated purity exceeding 99%, with a notable retention time shift from 11.0 minutes to 10.5 minutes after Cy5 conjugation (FIG. 3C). Additionally, we monitored the HPLC chromatogram of all major intermediates and final dendrimer conjugates at multiple wavelengths [210 (dendrimer), 254 (aromatic), 331 (axitinib), and 650 nm (Cy5)] to detect any impurities related to trehalose, axitinib, or Cy5, suggesting highly pure compounds.

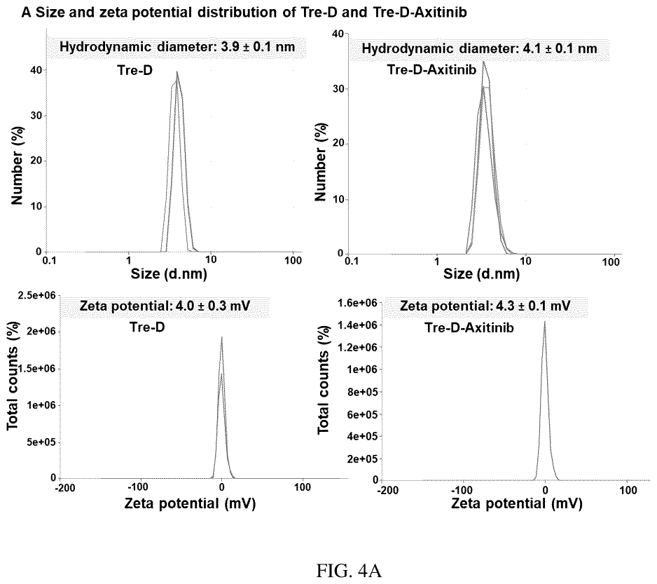

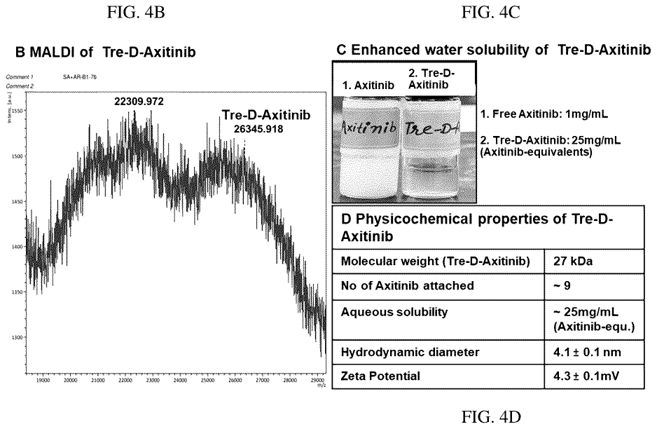

Dynamic light scattering (DLS) analysis revealed that Tre-D has a hydrodynamic diameter of approximately 4 nm in the range of renal filtration and has a nearly neutral zeta potential (~+4.0 mV) (FIGS. 4A and 4D). The conjugation of Axitinib did not significantly alter the size (~4.1 nm) and zeta potential (~+4.3 mV) of Tre-D-Axitinib conjugate. Theoretical molecular weight of Tre-D-Axitinib is 26,556 Da and the MALDI-TOF analysis confirmed the molecular weight of Tre-D-Axitinib (FIGS. 4B and 4D). MALDI-TOF analysis of Tre-D-Axitinib (matrix: DHB, linear positive mode) produced a broad molecular ion envelope, which is typical for high-molecular-weight dendrimer-drug conjugates. Such broadness arises from the inherent polydispersity of the conjugation process and the variability in ionization/desorption efficiency for large, heterogeneous molecules in MALDI. These factors can also contribute to a slight apparent underestimation of molecular weight in the spectrum. In our data, the dominant peak cluster centered near ~26.3 kDa is in close agreement with the calculated molecular weight of 26.56 kDa for Tre-D-Axitinib, supporting the expected composition and confirming the successful synthesis of the conjugate. Moreover, the conjugation of Axitinib on dendrimer significantly enhanced the aqueous solubility of Axitinib, which is almost insoluble in water (0.2 μg/mL; FIGS. 4C and 4D). The solubility of Tre-D-Axitinib based on Axitinib equivalent is ~25mg/mL in water which is almost 125,000 folds more than the free Axitinib.

The shelf stability of the Tre-D-Axitinib formulation was evaluated in PBS at 4 °C, and room temperature (25 °C) over a 30-day period. Remarkably, the formulation maintained stability throughout this duration, with purities exceeding 99%, as confirmed by HPLC analysis (PDA detector; 331 nm). Additionally, no shifts in retention time were observed, nor was there any detectable release of Axitinib from the formulation, demonstrating the conjugate's stability under these conditions. We then evaluated the stability of Tre-D-Axitinib at physiological conditions (37 oC, PBS buffer at pH 7.4) over a period of 30 days. No degradation or release of Axitinib was detected, as shown by the HPLC chromatograms (PDA detector; 331 nm) at various time points. This study suggests the structural integrity of the Tre-D-Axitinib conjugate during blood circulation. This non-cleavable conjugate is designed to be effective without the need of Axitinib to be released from the dendrimer. This design prevents premature drug release, reducing the risk of off-target effects and potential toxicity.

### In Vitro Cellular Uptake and VEGFR2 Inhibition Potential of Tre-D-Axitinib

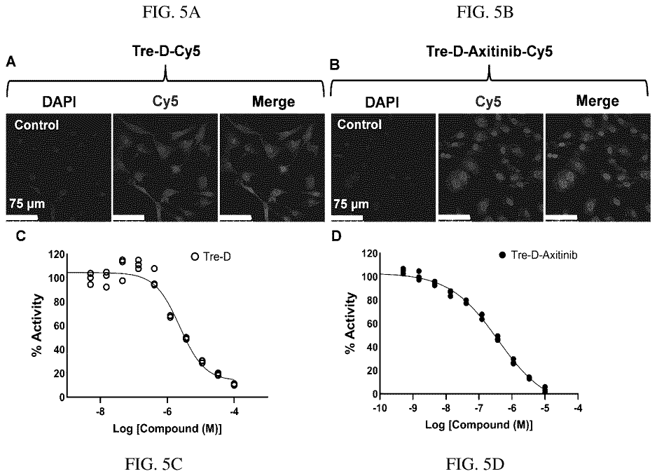

Before proceeding with the in vivo studies, we first assessed the cellular uptake of Tre-D and Tre-D-Axitinib in HUVECs. Fluorescently labeled conjugates, Tre-D-Cy5 and Tre-D-Axitinib-Cy5, were utilized to evaluate the impact of Axitinib conjugation on the uptake of Tre-D in HUVECs. Both Tre-D and Tre-D-Axitinib effectively internalized into the cells, and the conjugation of the hydrophobic Axitinib did not affect the intracellular uptake of Tre-D (FIGS. 5A & 5B). Subsequently, we examined the VEGFR2 inhibition potential of Tre-D and the Tre-D-Axitinib conjugate. Interestingly, the Tre-D platform itself exhibited some VEGFR2 inhibition activity, with an IC50 of approximately 4 μM (FIG. 5C) which aligns with literature evidence suggesting that trehalose can inhibit VEGF-induced angiogenesis and vascular proliferation. The VEGFR2 inhibition activity of Tre-D-Axitinib was observed to be in nanomolar range (~260 nM; FIG. 5D). Considering broad-spectrum inhibition of Axitinib as multiple tyrosine kinases, it is likely that Tre-D-Axitinib exerts its biological effects through a combination of VEGF receptor suppression and interference with other kinase-mediated signaling pathways.

### Tre-D-axitinib Conjugates Reduce Angiogenic Events in HUVECS

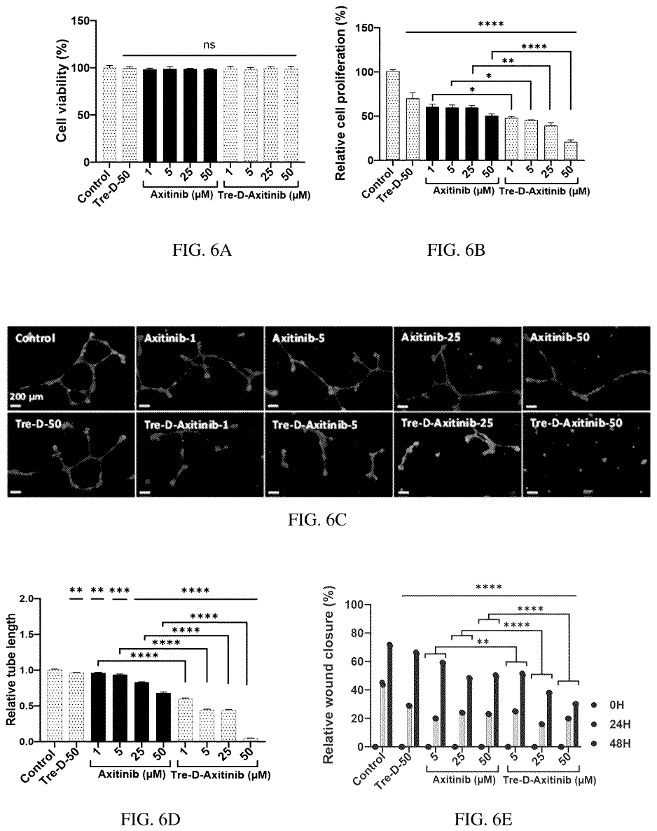

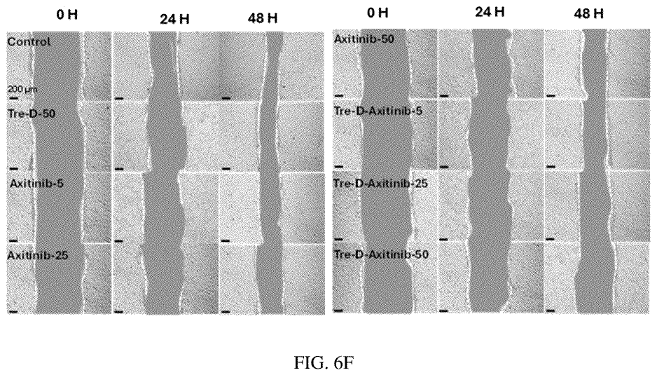

Before proceeding with the efficacy studies, we first evaluated the in vitro cellular compatibility of Tre-D control and Tre-D-Axitinib in HUVEC cells (FIG. 6A), and RAW macrophages. The results clearly suggest that both control dendrimer (Tre-D) and the Axitinib conjugate (Tre-D-Axitinib) display excellent cytocompatibility at all tested concentrations (1, 5, 25, and 50 μM). Cell proliferation is crucial in the development of ROP, which is characterized by excessive retinal vascularization in premature infants. The increased proliferation leads to the aberrant growth of the blood vessels, resulting in serious visual impairment. Previously, it has been shown that Axitinib has potential anti-proliferation effect on the cells by selectively inhibiting VEGF receptors signaling and thereby impairing endothelial cell function and abnormal vascular development. Our in vitro results indicate that both Axitinib and Tre-D-Axitinib display anti-proliferation effect in a dose dependent manner. In comparison to the untreated control cells (100 % relative cell proliferation), the cell proliferation for free Axitinib and Tre-D-Axitinib at 1, 5, 25 and 50 μM was ~60.7, ~59.7, ~59.6, ~50.5 % and ~48.2, ~45.7, ~39.2, ~20.9 % respectively. The cell proliferation decreased to ~3 and ~5 folds for Axitinib and Tre-D-Axitinib at 50 μM. Moreover, Tre-D-Axitinib is significantly more effective than the free drug (FIG. 6B). Upon comparing the anti-proliferative effects of Axitinib and Tre-D-Axitinib, we observed a significant difference in cell proliferation rates. Specifically, Tre-D-Axitinib at concentrations of 1, 5, 25, and 50 μM exhibited approximately ~1.2, ~1.3, ~1.5, and ~2.4-fold greater inhibition of cell proliferation, respectively, compared to the free Axitinib. These findings indicate that Tre-D-Axitinib demonstrates markedly enhanced anti-proliferative activity compared to both free Axitinib and the dendrimer alone.

Next, we evaluated the effect of Tre-D-Axitinib on angiogenesis in HUVECs. It is evident from the fluorescent micrographs that even higher concentrations of free Axitinib (Axi-50) could only decrease the tube length to one-half (less than ~2 fold change) with respect to the control group, as quantified by ImageJ software. However, Tre-D-Axitinib showed a reduction in tube length in a dose dependent manner. Interestingly, the treatment with Tre-D-Axitinib at 50 μM showed a dramatic decrease in tube length (~25 folds) suggesting its potential anti-angiogenic effect on the HUVECs (FIGS. 6C and 6D). Comparing the efficacy of free Axitinib and Tre-D-Axitinib, the latter exhibited approximately 1.6, 2.1, 1.9, and 13.6-fold reductions in relative tube length at concentrations of 1, 5, 25, and 50 μM, respectively (FIGS. 6C and 6D). These results indicate that Tre-D-Axitinib possesses superior anti-angiogenic potential in HUVEC cells compared to free Axitinib and naked Tre-D, highlighting its use as a therapeutic agent in the management of ROP.

Tre-D-Axitinib exhibited a greater therapeutic effect than free Axitinib when treated at the same molar drug concentration. This enhanced efficacy is likely attributable to the dendrimer's multivalent architecture, which presents multiple Axitinib moieties in close spatial proximity, enabling simultaneous engagement with multiple VEGFR2 receptors on the endothelial cell surface. Such multivalent binding (avidity) can enhance receptor clustering, prolong receptor occupancy, and more effectively inhibit VEGFR2-mediated signal transduction, potentially without requiring intracellular release of Axitinib. In this context, cleavage of Axitinib from the Tre-D nanocarrier may not be essential for biological activity. The improved performance of Tre-D-Axitinib at equivalent molar concentrations further supports a multivalent binding mechanism, which could provide a distinct pharmacological advantage over the monovalent binding of free Axitinib.

### Tre-D-Axitinib Inhibits Cell Migration or Wound Healing in HUVECS

Cell migration is critical in the pathogenesis of ROP, specifically impacting angiogenesis and vascularization within the retina. Abnormal endothelial cell migration is a crucial element in ROP pathology, influencing the balance of normal and pathological vascular development. The uncontrolled cell migration in ROP leads to the development of unorganized complexes of blood vessels into the vitreous cavity, further causing retinal detachment and vision impairment. We next evaluated the impact of Tre-D-Axitinib on cell migration using HUVECs. The qualitative microscopy results suggested a distinct difference between the anti-cell migration effect of the Tre-D control, free Axitinib and the Tre-D-Axitinib conjugate at different time points. At 24 h, as compared to the control group (~44 %), free Axitinib and Tre-D-Axitinib at 5, 25, and 50 μM, the cell migration was found to be ~20, ~24, ~23 % and ~25, ~16, ~20 %, respectively. Furthermore, at 48 h, the cell migration or wound closure rate was observed to be ~71 % (control), and ~59%, ~48%, and ~50% for free drug at tested concentrations of 5, 25, and 50 μg/mL, respectively. In contrast, Tre-D-Axitinib at the same concentrations (5, 25, and 50 μg/mL) showed reduced migration rates of approximately ~51%, ~38%, and ~30%, respectively. This corresponds to a ~1.2, ~1.3, and ~1.7-fold decrease in cell migration for Tre-D-Axitinib compared to free Axitinib. These data suggest that Tre-D-Axitinib treated HUVECs were less proficient in migrating compared to untreated and Tre-D treated control and free drug (FIGS. 6E and 6F).

### Qualitative and Quantitative Biodistribution of Systemically Administered Tre-D and Tre-D-Axitinib in OIR Mouse Model

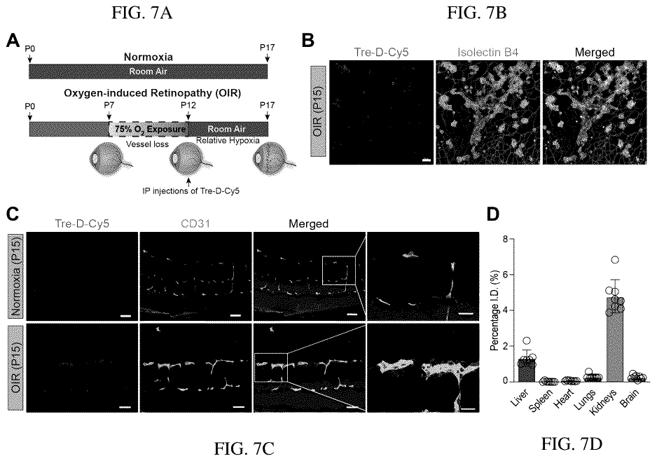

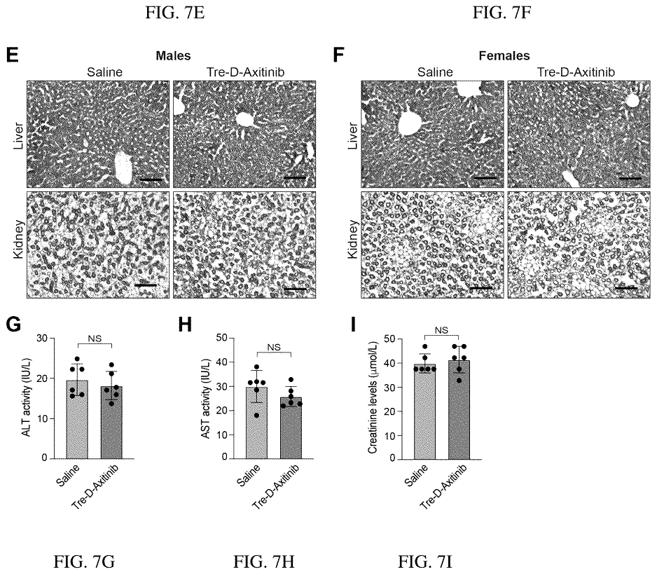

Developing systemic therapeutic approaches for retinal diseases is the focus of ongoing research due to the drawbacks associated with intravitreal delivery approaches. To evaluate the significance of Tre-D as a delivery mechanism for retinal targeting when IP administered in proliferative retinopathies, we utilized a mouse model of OIR. In this model, mouse pups are exposed to hyperoxia (75% oxygen) from P7-P12, which induces vessel regression and disrupts normal radial vascular development. Upon returning to room air at P12, the previously avascular retinal regions become hypoxic, triggering the upregulation of angiogenic factors and leading to retinal NV. (FIG. 7A). The neovascular phase of this model corresponds to the symptoms of proliferative DR and ROP in humans. We exposed mouse pups to OIR before administering Tre-D-Cy5 intraperitoneally at P12. At P15, we enucleated the eyes, extracted the retina, stained it with isolectin B4, and examined the retinas for Cy5 staining. We observed enhanced accumulation of Cy5 signal in neovascular tufts (FIGS. 7B & 7C), which suggested that we could use IP injections of Tre-D as a delivery vehicle to target pathological angiogenesis in proliferative retinopathies. We also administered Tre-D-Cy5 to normoxic mice and examined the localization of Tre-D-Cy5 in the retinas of normoxic mice at P15, noting minimal colocalization of Tre-D-Cy5 within the vascular cells (FIG. 7C). The observed colocalization appears to be confined to the endothelial cells and does not extend outside the vessels in normoxic mice. Before assessing the effect of Tre-D-Axitinib-Cy5 on pathological retinal NV, we evaluated the quantitative distribution of Tre-D-Axitinib-Cy5 in all major organs of OIR-treated mice at P17 following systemic administration at P12. Given that off-target localization and accumulation of nanoparticles poses a significant concern for their clinical application, we thoughtfully designed the Tre-D-Axitinib to be within the size range suitable for renal filtration to prevent unwanted accumulation in major organs. The biodistribution of Tre-D-Axitinib-Cy5 was analyzed in all major organs, including the kidneys, liver, brain, heart, lungs, and spleen (FIG. 7D). The animals were perfused with PBS to reduce interference from residual blood and dendrimers retained within the vasculature. The data suggested a minimal off-target uptake that was <1% in all major organs except kidneys which was ~5%. Furthermore, at P17, the liver and kidney sections of the control and Tre-D-Axitinib-treated groups showed no significant damage or difference (FIGS. 7E & 7F). Additionally, no significant differences were observed in liver and kidney enzyme levels, including Alanine aminotransferase (ALT), aspartate aminotransferase (AST), and creatinine, compared to saline-treated animals (FIG. 7G-I). These results suggest that the dendrimers are non-toxic to the liver and kidneys in both male and female mice. The combination of targeted retinal uptake and efficient clearance from off-site locations highlights Tre-D as a nanoplatform for developing retina-targeted therapies for a range of ocular disorders.

### Systemically Administered Tre-D-axitinib Reduces Pathological Retinal NV in OIR Mouse Model

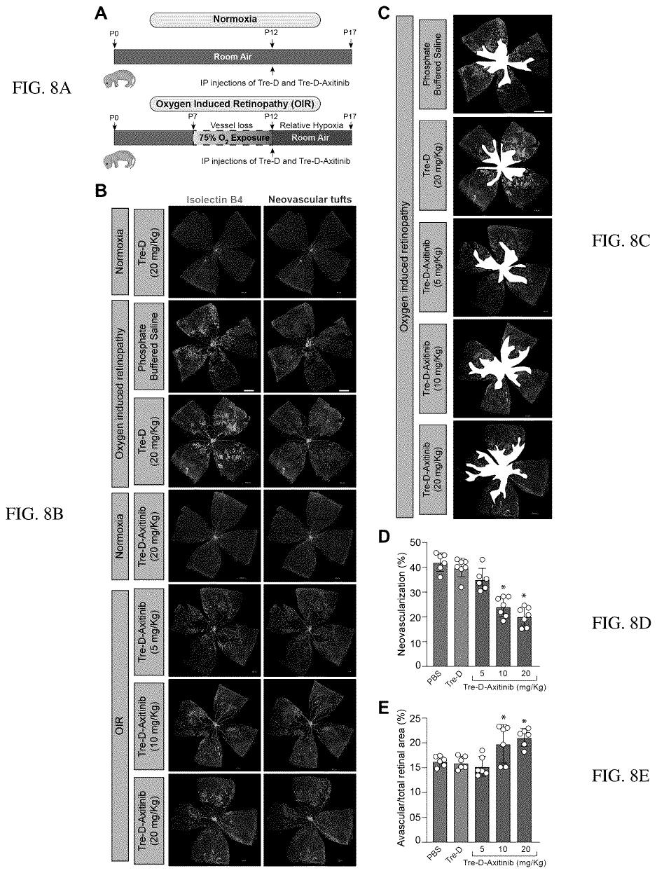

To find out how Tre-D-Axitinib affects abnormal retinal NV in proliferative retinopathies, we investigated the role of IP injection of Tre-D-Axitinib into normoxic retinas and a mouse model of OIR. We evaluated how it affected neovascular tufts and avascular areas in hypoxic retinas (FIG. 8A). We observed that single IP injection of Tre-D-Axitinib at P12, while having no effect on normoxic retinas, significantly reduced OIR-induced retinal NV and showed a reduction in tufts formation or anastomoses in hypoxic retinas when compared to PBS or Tre-D treated animals (FIGS. 8B & 8D). In addition, we also observed that IP injection of Tre-D-Axitinib resulted in decreased retinal vascularization and led to more widespread area of vaso-obliteration within the ischemic retina (FIGS. 8C & 8E). Notably, even though control Tre-D platform demonstrated some VEGFR2 inhibition activity and reduced cell proliferation to some extent in vitro, that did not translate in vivo at tested concentration. Retinal NV is a clinical symptom of various proliferative retinopathies, including ROP, DR, and the wet form of macular degeneration. If untreated, retinal NV will ultimately result in visual loss. Intravitreal injections of anti-angiogenic agents are frequently employed to manage these proliferative retinopathies. Intravitreal injections were often associated with endothalmitis, increased intraocular pressure, retinal detachment, vitreous hemorrhage, cataract formation, macular edema, corneal abrasions, and pain or discomfort. Recent research shows that diabetic patients who received intravitreal anti-VEGFA injections experienced systemic adverse effects. Consequently, ongoing research is being conducted to design therapeutic strategies that can replicate the effects of intravitreal injections via alternative administration routes. These present findings imply that systemic Tre-D-Axitinib administration can be employed to treat ischemic retinopathies, as well as to mitigate the disadvantages of intravitreal injections. These results hold strong promise for developing therapeutic approaches aimed at managing or preventing retinal disorders associated with pathological neovascularization.

### Tre-D-axitinib Attenuates VEGFA-Induced Angiogenic Events in HRMVECs

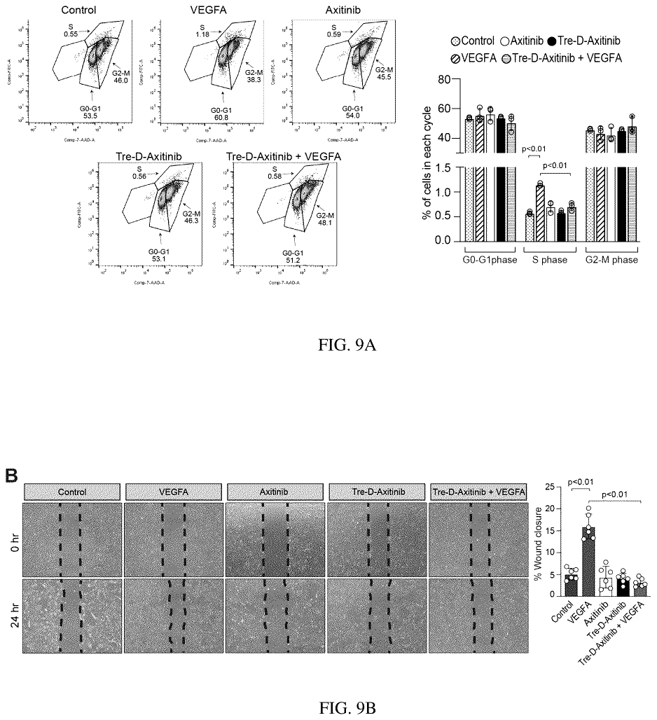

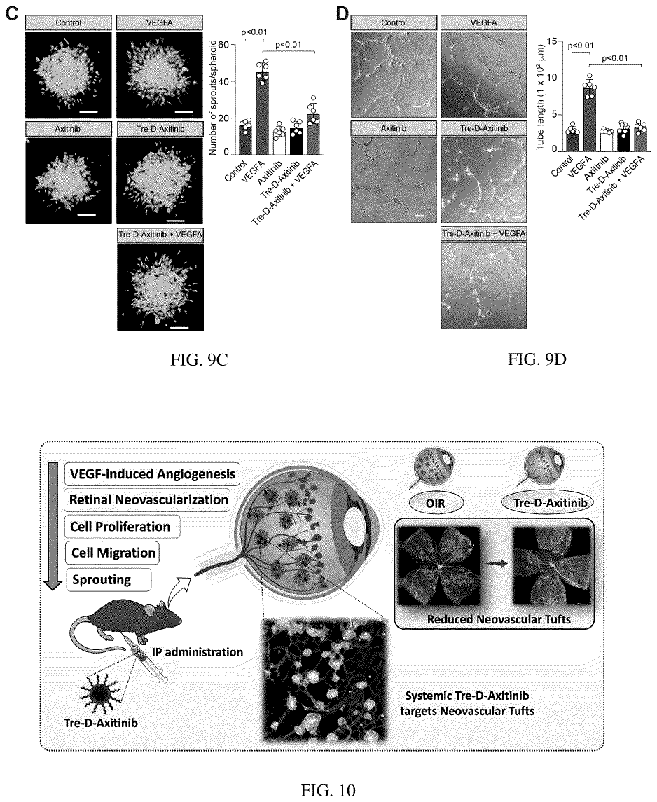

OIR is a well-established model in which elevated VEGFA expression has been documented in the retina and anti-VEGFA therapies have been shown to effectively inhibit NV in several diseases. To investigate the functional role of Tre-D-Axitinib in VEGFA-driven angiogenic processes, we examined its effect (20 ng/mL) on VEGFA-induced proliferation, migration, sprouting, and tube formation in HRMVECs. The FITC BrdU assay was employed to assess the impact of Tre-D-Axitinib on VEGFA-induced cell proliferation. As expected, VEGFA treatment significantly increased HRMVEC proliferation compared to vehicle control. However, pretreatment with Tre-D-Axitinib markedly suppressed this VEGFA-induced proliferation, as observed in the Tre-D-Axitinib+VEGFA group (FIG. 9A). The effect of Tre-D-Axitinib on VEGFA-induced cell migration was evaluated using a wound healing assay. VEGFA promoted HRMVEC migration, whereas pretreatment with Tre-D-Axitinib significantly inhibited this effect, as shown in the Tre-D-Axitinib+VEGFA group (FIG. 9B). The effects of Tre-D-Axitinib on tip cell formation/sprouting and tube formation were evaluated using a 3D angiogenic spheroid assay and a 2D Matrigel assay, respectively. VEGFA stimulation promoted tip cell formation/sprouting and tube formation in HRMVECs, whereas pretreatment with Tre-D-Axitinib significantly reduced these VEGFA-induced angiogenic responses, as observed in the Tre-D-Axitinib+VEGFA group (FIGS. 9C and 9D). No significant effects of Axitinib or Tre-D-Axitinib alone were observed on HRMVEC proliferation, migration, sprouting, or tube formation (FIG. 9A-D). In retinal endothelial cells, VEGFA modulates physiological and pathological angiogenesis in proliferative retinopathies through VEGF receptors activation. Previous research indicates that Axitinib inhibits VEGF receptors as well as other tyrosine kinase receptors. In our current study, we observed that Tre-D-Axitinib inhibits VEGFA-induced angiogenic signaling, suggesting that its mechanism of action may involve inhibition of VEGF receptor activation. Given the established role of VEGF signaling in promoting retinal angiogenesis, and its upregulation in the vasculature of patients with proliferative retinopathies, it is plausible that VEGF receptor blockade contributes to the anti-angiogenic effects observed with Tre-D-Axitinib. However, due to Axitinib's known broad-spectrum activity, it remains possible that Tre-D-Axitinib's effects are mediated through a combination of VEGF receptor inhibition and modulation of other tyrosine kinase pathways. Furthermore, it has been demonstrated that ischemia and hypoxia (in this case OIR) damage retinal blood vessels and disrupt blood-retinal barrier (BRB) integrity. The disruption of BRB integrity enables Tre-D-Axitinib to preferentially accumulate in ischemic retinas, where it subsequently inhibits the activation and downstream signaling of its target receptor tyrosine kinases. This targeted delivery demonstrates the NV-specific therapeutic potential of Tre-D-Axitinib in ischemic retinal tissue. Together, these findings support the use of Tre-D-Axitinib as a targeted therapeutic for ischemic retinal diseases.

### Conclusions

Current interventions for proliferative retinopathies, while effective, face substantial limitations. Intravitreal anti-VEGFA therapies are invasive, require frequent administration, and are associated with ocular complications and systemic adverse effects. Additionally, these therapies often fail to achieve selective targeting of neovascular tufts, limiting their therapeutic efficacy. No organic nanoparticles have yet demonstrated the ability to specifically localize within aberrant neovascular tufts at retinal pathology sites after systemic delivery. Tre-D-Axitinib addresses these gaps by providing a systemic, minimally invasive alternative that combines the selective targeting capabilities of Tre-D with the potent anti-angiogenic effects of Axitinib. Tre-D is designed to have inherent NV targeting abilities without the need of any additional targeting ligands, limiting post-synthetic modifications. Our findings demonstrate that Tre-D-Axitinib significantly reduces pathological NV while leading to increased vaso-obliteration in the ischemic retina. These findings suggest that Tre-D-Axitinib, in addition to reducing pathological retinal NV, may also affect vascular repair. The high aqueous solubility, biocompatibility, and scalable synthesis of Tre-D further enhance its clinical viability, presenting a robust alternative to existing intravitreal therapies. The development of Tre-D-Axitinib represents a significant step forward in nanomedicine, offering a transformative solution to the challenges associated with the current treatment of ischemic retinopathies.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for delivering one or more agents to a retina or cornea of a subject, comprising administering to the subject a composition comprising a trehalose-surfaced dendrimer (Tre-D) conjugated to the one or more agents.

2. The method of claim 1, wherein the Tre-D targets aberrant neovascular tufts in a retina or aberrant blood vessels in a cornea of the subject.

3. The method of claim 1, wherein the one or more agents are conjugated to an outer surface of the Tre-D.

4. The method of claim 3, wherein the one or more agents are conjugated to the outer surface via a covalent bond.

5. The method of claim 3, wherein the one or more agents are conjugated to the outer surface via noncovalent interactions.

6. The method of claim 1, wherein the one or more agents comprises a vascular endothelial growth factor (VEGF) inhibitor.

7. The method of claim 1, wherein the one or more agents comprises one or more of an anti-angiogenic agent, a tyrosine kinase inhibitor, an anti-cancer agent, a steroid, an anti-inflammatory agent (NSAID), and an anti-infective agent.

8. The method of claim 7, wherein the tyrosine kinase inhibitor is one or more of axitinib, lenvatinib, dasatinib, tivozanib, cabozantinib, sorafenib, regorafenib, anlotinib, brivanib, su1498, vorolanib, vatalanib, pazopanib, and vandetanib.

9. The method of claim 7, wherein the NSAID is one or more of aspirin, tolmetin, naproxen, bromfenac, ketorolac, nepafenac, ibuprofen, piroxicam, mefenamic acid, meloxicam, celecoxib, fenoprofen, sulindac, oxaprozin, etodolac, indomethacin, diclofenac, ketorolac, neparenac, and bromfenac.

10. The method of claim 1, wherein the one or more agents is present in the composition in an amount of 0.1-80 wt %.

11. The method of claim 1, wherein the Tre-D is administered via systemic, intravitreal, suprachoroidal, periocular, subconjunctival, intracameral, intraperitoneal, topical, oral, transscleral, subretinal, or implantable device administration.

12. A method for treating an ocular disorder in a subject in need thereof, comprising administering to the subject a therapeutically effective amount of a trehalose-surfaced dendrimer (Tre-D).

13. The method of claim 12, wherein the ocular disorder is selected from the group consisting of diabetic retinopathy, age related macular degeneration, proliferative retinopathies, retinitis pigmentosa, Stagardt's disorder, ocular histoplasmosis, choroidal neovascularization, corneal neovascularization, ocular inflammation uveitis, retinopathy of prematurity (ROP), retinal vein occlusion (RVO), hypertensive retinopathy, retinal angiomatomous proliferation, glaucoma, retinal detachment, and macula edema.

14. The method of claim 12, wherein the Tre-D targets aberrant neovascular tufts in a retina of the subject.

15. The method of claim 12, wherein the Tre-D is conjugated to one or more therapeutic agents suitable for treating the ocular disorder.

16. The method of claim 15, wherein the one or more therapeutic agents are conjugated to an outer surface of the Tre-D.

17. The method of claim 16, wherein the one or more therapeutic agents are conjugated to the outer surface via a covalent bond.

18. The method of claim 16, wherein the one or more therapeutic agents are conjugated to the outer surface via noncovalent interactions.

19. The method of claim 15, wherein the one or more therapeutic agents comprises a vascular endothelial growth factor (VEGF) inhibitor.

20. The method of claim 15, wherein the one or more therapeutic agents comprises one or more of an anti-angiogenic agent, a tyrosine kinase inhibitor, an anti-cancer agent, a steroid, an anti-inflammatory agent (NSAID), and an anti-infective agent.

21. The method of claim 20, wherein the tyrosine kinase inhibitor is one or more of axitinib, lenvatinib, dasatinib, tivozanib, cabozantinib, sorafenib, regorafenib, anlotinib, brivanib, su1498, vorolanib, vatalanib, pazopanib, and vandetanib.

22. The method of claim 20, wherein the NSAID is one or more of aspirin, tolmetin, naproxen, bromfenac, ketorolac, nepafenac, ibuprofen, piroxicam, mefenamic acid, meloxicam, celecoxib, fenoprofen, sulindac, oxaprozin, etodolac, indomethacin, diclofenac, ketorolac, neparenac, and bromfenac.

23. The method of claim 15, wherein the one or more therapeutic agents is present in the composition in an amount of 0.1-80 wt %.

24. The method of claim 12, wherein the Tre-D is administered via systemic, intravitreal, suprachoroidal, periocular, subconjunctival, intracameral, intraperitoneal, topical, oral, transscleral, subretinal, or implantable device administration.
